(19) United States
(12) Patent Application Publication
(10) Pub. No.: US 2026/0345693 A1
(43) Pub. Date: Nov. 5, 2026

(54) TARGETED PROBIOTICS AND PEPTIDES FOR REDUCTION OR AUGMENTATION OF BACTERIAL-SELF ANTIGEN CROSS-REACTIVE MEMORY T-CELLS

(71) Applicant: UNIVERSITY OF WASHINGTON, SEATTLE, WA (US)

(72) Inventors: Mary L. Disis, SEATTLE, WA (US); Denise Cecil, SEATTLE, WA (US)

(73) Assignee: UNIVERSITY OF WASHINGTON, SEATTLE, WA (US)

(21) Appl. No.: 19/125,613

(22) PCT Filed: Oct. 27, 2023
(86) PCT No.: PCT/US23/78034

§ 371 Date: Apr. 29, 2025

Related U.S. Application Data
(60) Provisional application No. 63/381,675, filed on Oct. 31, 2022.

(51) Int. Cl. A61K 35/741; A61P 43/00

(52) U.S. Cl. CPC A61K 35/741; A61P 43/00

(57) ABSTRACT

Specific organisms derived from the gut microbiome that we have found rarely or never share homologies with tumor antigens are used to modulate bacteria-tumor antigen cross-reactive T-cells. Bacteria with low to no homologies can be safely enriched in the gut and prevent these cross-reactive T-cells from expansion. This precision modulation of the gut microbiome reduces tumor growth and increases the efficacy of immune checkpoint inhibitor therapy in a model of triple negative breast cancer. Moreover, the use of specific organisms derived from the gut microbiome that share homologies with self-proteins can be used to modulate the autoimmune inflammatory response. This precision modulation of the gut microbiome can be used to elicit a Type 2 immune response.

This application claims benefit of U.S. provisional patent application No. 63/381,675, filed Oct. 31, 2022, the entire contents of which are incorporated by reference into this application.

### ACKNOWLEDGEMENT OF GOVERNMENT SUPPORT

This invention was made with government support under Grant No. W81XWH-11-1-0760, awarded by the Department of Defense. The government has certain rights in the invention.

### REFERENCE TO A SEQUENCE LISTING

The content of the XML file of the sequence listing named “UW80_seq”, which is 5 kb in size, created on Oct. 25, 2023, and electronically submitted herewith the application, is incorporated herein by reference in its entirety.

### BACKGROUND

High levels of Type I T-cells, both cytotoxic and T-helper cells (Th1) are needed to eradicate breast cancer. Few cancers induce Type I immunity with the exception of tumors that contain a high mutational burden. The majority of solid tumor patients develop a Type II immune response to commonly recognized tumor antigens, which are aberrantly expressed non-mutated tumor associated proteins. The development of a Type II T-cell immune signature is an early event in many common solid tumors, including breast cancer, and portends more aggressive disease (1, 2). We found that within the amino acid sequence of non-mutated tumor associated antigens are class II binding epitopes that preferentially elicit a T-helper 1 interferon-gamma (g) (Th1) or an IL-10 T-helper-2 (Th2) response and are selective for that CD4 phenotype (3). The Th2 have high functional avidity for the non-mutated tumor antigen, secreting high levels of IL-10 and IL-6 after stimulation with very low doses of antigen in contrast to the lower avidity Th1. These Th2 responses will dominate the tumor microenvironment as Type II cytokines are a potent inhibitor of cytotoxic T-cells.

There remains a need to replace treatment regimens for cancer with ones that are more effective, less toxic, and impact survival.

### SUMMARY

The material described herein meets these needs and others by providing compositions and methods for enhancing immunity to a tumor antigen in a subject, as well as for slowing tumor growth in a subject in need thereof. In some embodiments, these methods comprise administering to the subject an effective amount of a composition comprising a plurality of bacteria that exhibit less than 50% amino acid sequence homology to non-mutated tumor antigens (bacteria^{low}). In some embodiments, the plurality of bacteria^{low} are selected from: Lactobacillus acidophilus, Ruminococcus albus, Ruminococcus gnavus, Dorea formicigenerans, Eubacterium siraeum, Bacteroides stercoris, Parabacteroides distasonis, Bifidobacterium breve, Lactobacillus delbrueckii, Odoribacter splanchnicus, Akkermansia muciniphila, Desulfovibrio piger, Mitsuokella multacida, Bifidobacterium catenulatum, Staphylococcus saprophyticus, Bacteroides vulgatus, Bifidobacterium pseudocatenulatum, Bifidobacterium angulatum, Bifidobacterium bifidum, Campylobacter hominis, Clostridium leptum, Clostridium methylpentosum, Coprococcus catus, Eubacterium ventriosum, Ruminococcus bromii, Victivallis vadensis, Bacteroides eggerthii, Bacteroides helcogenes, Granulicatella adiacens, Peptoniphilus harei In some embodiments, the plurality of bacteria^{low} comprise at least four bacteria selected from: R. albus, L. acidophilus, A. muciniphila, B. breve, O. splanchnicus and P. distasonis. In some embodiments, the plurality of bacteria^{low} comprise R. albus and L. acidophilus.

In some embodiments, the composition is free of bacteria exhibiting greater than 50% homology to tumor antigens identified in a blood sample obtained from the subject. In some embodiments, the method further comprises obtaining such a blood sample from the subject, and identifying bacterial-tumor antigen (BAC-TA) cross-reactive memory T-cells detected in the sample. In some embodiments, the composition is administered weekly for six weeks. In some embodiments, the composition is administered twice a week. In some embodiments, the composition is administered twice a month. In some embodiments, the composition is administered monthly. In some embodiments, the administration continues for at least two months. In some embodiments, the administration continues for at least 6, 8, 12, or 24 months.

In some embodiments, the effective amount is sufficient to effect a two-fold increase in gut microbiome bacteria^{low}, and/or a significant decrease in bacterial-tumor antigen (BAC-TA) cross-reactive memory T-cells, in a biological sample obtained from the subject. In some embodiments, the method enhances efficacy of a cancer treatment. In some embodiments, the cancer treatment comprises immunotherapy and/or chemotherapy. In some embodiments, the immunotherapy comprises administration of immune checkpoint inhibitor therapy, vaccine therapy, cytokine therapy, and/or oncolytic virus therapy. In some embodiments, the chemotherapy comprises administration of paclitaxel. Other chemotherapeutic agents, as well as other immunotherapeutic agents, known to those skilled in the art are likewise contemplated. Some representative examples of chemotherapeutic agents include, but are not limited to, cisplatin, carboplatin, pemtrexed, Adriamycin, Cytoxan. Some representative examples of immunotherapeutic agents include, but are not limited to, monoclonal antibodies, such as, for example, anti-HER2, anti-EGFR, and anti-VEGF. In some embodiments, the composition is administered prior to and/or concomitantly with immunotherapy or chemotherapy.

In some embodiments, the method enhances a type 1 immune response. In some embodiments, the method increases the relative amount of bacteria^{low} that can be detected in a biological sample obtained from the subject. Examples of biological samples include blood and stool samples.

In some embodiments, the composition comprises 1-100×10⁹ CFU of bacteria^{low}. In some embodiments, the composition comprises 1-50×10⁹ CFU of bacteria^{low}. In some embodiments, the composition comprises 1-10×10⁹ CFU of bacteria^{low}. In some embodiments, the composition comprises 1-5×10⁹ CFU of bacteria^{low}.

In some embodiments, the administration of the composition is oral, rectal, or intragastric. In some embodiments, the composition is delivered in the form of a suppository.

Also described is a composition comprising a plurality of bacteria that exhibit less than 50% homology to non-mutated tumor antigens (bacteria^{low}). In some embodiments, the plurality of bacteria^{low} are selected from: Lactobacillus acidophilus, Ruminococcus albus, Ruminococcus gnavus, Dorea formicigenerans, Eubacterium siraeum, Bacteroides stercoris, Parabacteroides distasonis, Bifidobacterium breve, Lactobacillus delbrueckii, Odoribacter splanchnicus, Akkermansia muciniphila, Desulfovibrio piger, Mitsuokella multacida, Bifidobacterium catenulatum, Staphylococcus saprophyticus, Bacteroides vulgatus, Bifidobacterium pseudocatenulatum, Bifidobacterium angulatum, Bifidobacterium bifidum, Campylobacter hominis, Clostridium leptum, Clostridium methylpentosum, Coprococcus catus, Eubacterium ventriosum, Ruminococcus bromii, Victivallis vadensis, Bacteroides eggerthii, Bacteroides helcogenes, Granulicatella adiacens, Peptoniphilus harei In some embodiments, the plurality of bacteria^{low} comprise at least four bacteria selected from: R. albus, L. acidophilus, A. muciniphila, B. breve, O. splanchnicus and P. distasonis. In some embodiments, the plurality of bacteria^{low} comprise R. albus and L. acidophilus. In some embodiments, the composition is substantially free of bacteria that exhibit greater than 50% homology to tumor antigens.

Also described is a method of ameliorating an autoimmune inflammatory response in a subject. In some embodiments, the method comprises administering to the subject an effective amount of a composition comprising a plurality of bacteria that exhibit at least 50% (high) homology to self-proteins (bacteria^{high}). In some embodiments, the plurality of bacteria^{high} are selected from: Escherichia coli, Enterococcus faecalis, Pseudomonas fluorescens, Pseudomonas putida, Pseudomonas aeruginosa, Shigella dysenteriae, Enterobacter cloacae, Lactococcus lactis, Clostridium perfringens, Klebsiella pneumoniae, Staphylococcus aureus, Enterococcus faecium, Rhodopseudomonas palustris, Shigella boydii, Shigella sonnei, Finegoldia magna, Clostridium asparagiforme, Collinsella aerofaciens, Fusobacterium ulcerans, Aerococcus viridans, Bacteroides ovatus, Clostridium botulinum, Enterococcus gallinarum, Eubacterium limosum, Faecalibacterium prausnitzii, Holdemania filiformis, Lactobacillus plantarum, Lactobacillus reuteri, Marvinbryantia formatexigens, Megamonas hypermegale, Streptococcus agalactiae, Streptococcus anginosus, Streptococcus dysgalactiae, and Streptococcus infantarius. In some embodiments, the plurality of bacteria^{high} exhibit at least 50% homology to self-proteins identified in a blood and/or stool sample obtained from the subject.

In some embodiments, the composition is administered weekly for six weeks to one year. In some embodiments, the composition is administered weekly for at least one year. In some embodiments, the composition is administered twice a week. In some embodiments, the composition is administered twice a month. In some embodiments, the composition is administered monthly. In some embodiments, the administration continues for at least two months. In some embodiments, the administration continues for at least 6, 8, 12, or 24 months.

In some embodiments, the effective amount is sufficient to effect a two-fold increase in gut microbiome bacteria^{high} in a biological sample obtained from the subject. Examples of biological samples include blood and stool samples. In some embodiments, the method further comprises obtaining a biological sample from the subject, and identifying self-protein reactive memory T-cells.

In some embodiments, the method elicits a type 2 immune response. In some embodiments, the method elicits an increase in the amount of IL-10 and/or IL-6 that can be detected in a biological sample obtained from the subject.

In some embodiments, the composition 1-100×10⁹ CFU of bacteria^{high}. In some embodiments, the composition comprises 1-50×10⁹ CFU of bacteria^{high}. In some embodiments, the composition comprises 1-10×10⁹ CFU of bacteria^{high}. In some embodiments, the composition comprises 1-5×10⁹ CFU of bacteria^{high}.

In some embodiments, the administration of the composition is oral, rectal, or intragastric. In some embodiments, the composition is delivered in the form of a suppository.

Also described is a composition comprising a plurality of bacteria that exhibit at least 50% amino acid sequence homology to self-proteins (bacteria^{high}). In some embodiments, the plurality of bacteria^{high} are selected from: Escherichia coli, Enterococcus faecalis, Pseudomonas fluorescens, Pseudomonas putida, Pseudomonas aeruginosa, Shigella dysenteriae, Enterobacter cloacae, Lactococcus lactis, Clostridium perfringens, Klebsiella pneumoniae, Staphylococcus aureus, Enterococcus faecium, Rhodopseudomonas palustris, Shigella boydii, Shigella sonnei, Finegoldia magna, Clostridium asparagiforme, Collinsella aerofaciens, Fusobacterium ulcerans, Aerococcus viridans, Bacteroides ovatus, Clostridium botulinum, Enterococcus gallinarum, Eubacterium limosum, Faecalibacterium prausnitzii, Holdemania filiformis, Lactobacillus plantarum, Lactobacillus reuteri, Marvinbryantia formatexigens, Megamonas hypermegale, Streptococcus agalactiae, Streptococcus anginosus, Streptococcus dysgalactiae, and Streptococcus infantarius.

In some embodiments, the plurality comprises at least four species of bacteria^{high} In some embodiments, the plurality comprises 6-10 species of bacteria^{high}.

In some embodiments, the subject to be treated in the above methods is human. In some embodiments, the subject is suspected of having, or has been diagnosed with cancer, such as breast cancer, or an autoimmune disease.

### BRIEF DESCRIPTION OF THE DRAWINGS

FIGS. 1A-1D show Human T-cell lines generated from IL-10-inducing epitopes are specific for the tumor antigen and cross-reactive to bacteria^{high}. (1A) Epitope induced IL-10-secreting precursor frequency to the indicated epitope presented as box and whisker plots. Each dot is the precursor frequency for an individual donor that demonstrated a significant IL-10 response. Mean (±SD) IL-10 corrected spots per well (CSPW) for T-cell lines specific for (1B) YB1p82 or (1C) BIRC5p13 stimulated with the line-generating epitope, corresponding recombinant protein or bacteria that share >50% sequence homology with the line-generating epitope. Negative controls include an irrelevant peptide (HIVp52) or protein (BSA) or bacteria that shares no sequence homology (R. albus). (D) Percent change in the indicted memory T-cell subset in the antigen-specific T-cell line compared to the autologous PBMC. **p<0.01; ***p<0.001; ****p<0.0001.

FIGS. 2A-2D show that BAC-TA cross-reactive T-cells traffic to mammary cancer and promote tumor growth. (2A) Epitope induced IL-10-secreting precursor frequency to the indicated epitope presented as box and whisker plots with Tukey outliers in naïve FVB mice (n=10), (2B) Total flux (±SEM) of fluorescently labeled T-cells measured in tumors from mice infused with P. aeruginosa-specific or naive T-cells (n=5). Representative fluorescent imaging quantified in panel. (2C) Mean (±SEM) IL-10 corrected spots per well (CSPW) for tumor infiltrating lymphocytes from mice infused with P. aeruginosa-specific T-cells stimulated with the indicated bacteria lysate. (2D) Mean (±SEM) tumor volume from mice infused with P. aeruginosa-specific (circles) or naïve (triangles) T-cells. **p=0.01, ****p<0.0001.

FIG. 3 shows that tumor infiltrating lymphocytes in spontaneous breast cancer are specific for bacteria^{high} Mean (±SEM) IL-10 corrected spots per well (CSPW) from spontaneous tumor infiltrating lymphocytes (bars with open circles) or spleen (bars with hatched circles) stimulated with the indicated bacterial lysate presented as box and whisker plots showing all points, horizontal line at median, whiskers min to max n=12 mice; **p<0.01.

FIGS. 4A-4C show that oral gavage of live bacteria resulted in increased bacterial counts of the gavaged species. (4A) Experimental design schematic. Number of bacteria detected in the stool of mice treated with (4B) R. albus and L. acidophilus or untreated and stool measured for those bacteria (4C) E. coli and P. aeruginosa or untreated presented as box and whisker plots showing all points, horizontal line at median, whiskers min to max. n=12 mice/group.

FIG. 5 shows that treatment with L. acidophilus and R. albus can enhance vaccine immunogenicity. Mean (±SEM) IFN-g corrected spots per well (CSPW) for mice immunized with the indicated vaccine and treated with the indicated bacteria. n=8 mice/group; *p<0.05.

FIGS. 6A-6B show that mammary tumors in the C3T are resistant to immunotherapy and chemotherapy. Mean (±SEM) tumor volume from untreated mice (∘) from a M6 implant mouse model treated with (6A) anti-PDL1 (▪) or (6B) paclitaxel (Δ). *p<0.05.

FIGS. 7A-7D show that tumor antigen epitopes with elevated IL-10-secreting T-cell precursor frequencies share high sequence homology with epitopes from bacteria in the gut microbiome. (7A) IL-10 secreting T-cell precursor frequency of all tumor antigen epitopes (n=84) presented as box and whisker plots for healthy volunteer (n=21) or cancer (n=20) donors. The number of distinct species of (7B) bacteria (7C) fungus and (7D) viruses sharing >50% homology with a tumor antigen epitope.

FIGS. 8A-8B show that there is no significant association between the relative start location or the number of amino acid mutations near an epitope and adjusted IL10 spot count factor of any epitope. The (8A) corresponding relative start location, (8B) number of non-synonymous amino acid mutations within ten amino acids and the adjusted IL-10 spot count factor. A locally estimated scatterplot smoothing (loess) line is drawn to show the smoothed mean and the shaded area corresponds to the 95% confidence interval for the smoothed mean. Those epitopes found in the top precursor frequency quintile are in hatched circles, those in the middle quintiles are in open circles and those in the bottom quintile are in cross-hatched circles.

FIGS. 9A-9F show that T-cell lines generated from IL-10-inducing tumor antigen epitopes are cross-reactive to bacteria with high sequence homology, express cytokines and genes associated with intestinal intraepithelial lymphocytes and are of a memory phenotype. Mean (±SD) IL-10 corrected spots per well (CSPW) for T-cell lines specific for (9A) Yb1-p82, (9B) BIRC5-p13 and (9C) PRL3-p104 stimulated with the line-generating epitope, corresponding recombinant protein or bacteria that share >50% sequence homology with the line-generating epitope. Negative controls include an irrelevant peptide (HIVp52) or protein (BSA) or a bacteria that shares no sequence homology (R. albus). (9D) Cytokine concentration (pg/ml) measured from the cell culture supernatant of antigen-specific T-cell lines presented as a heat map. (9E) Gene expression represented as fold-change for the bacterial-tumor antigen cross reactive T-cell lines compared to autologous CD3+ T cells presented as box plots, vertical line at median, for all three cultures. (9F) Percent change in T-cell memory phenotype in the T-cell lines as compared to autologous CD3+ T-cells presented as box plots, horizontal line at median, for the CD4 (left bars) or CD8 (right bars) subset. CM=central memory, EM=effector memory. *p<0.05, **p<0.01.

FIGS. 10A-10E show that murine T-cell lines generated from the same IL-10-inducing tumor antigen epitopes observed in humans recapitulated the phenotype of the human T-cell lines. Mean (±SD) IL-10 corrected spots per well (CSPW) for T-cell lines specific for (10A) Yb1-p82 or (10B) BIRC5-p13 stimulated with the line-generating epitope, corresponding recombinant protein or bacteria that share >50% sequence homology with the line-generating epitope. Negative controls include an irrelevant peptide (HIVp52) or protein (BSA) or a bacteria that shares no sequence homology (R. albus). (10C) Cytokine concentration (pg/ml) measured from the culture supernatant of antigen-specific T-cell lines presented as a heat map. (10D) Gene expression represented as fold-change for the bacterial-tumor antigen cross reactive T-cell lines compared to general CD3+ T cells presented as box plots, vertical line at median, for all cultures. (10E) Percent change in T-cell memory phenotype in the epitope-specific T-cell lines as compared to general CD3+ T cells presented as box plots, horizontal line as median, for the CD4 (left bars) or CD8 (right bars) subset. CM=central memory, EM=effector memory. *p<0.05, ****p<0.001.

FIGS. 11A-11B show that significantly more TCRb were common between intestinal intraepithelial lymphocytes and tumor infiltrating lymphocytes than splenocytes and tumor infiltrating lymphocytes. (11A) Venn diagram of the number of TCRb sequences shared between intestinal intraepithelial lymphocytes (IEL; upper circle in each trio) and tumor infiltrating lymphocytes (TIL; left circles) and/or spleen (right circles). (11B) Fold change of the number of shared TORb in IEL, TIL and spleen for each mouse normalized to the number of shared TCRb between TIL and spleen. n=7 mice; ***p<0.001.

FIGS. 12A-12F show that Pseudomonas aeruginosa-specific T-cells traffic to breast cancer and promote tumor growth. (12A). Mean (±SD) IL-10 corrected spots per well (CSPW) for T-cell lines specific for P. aeruginosa stimulated with the line-generating bacteria, homologous epitope Yb1-p82 and recombinant protein Yb1. Negative controls include an irrelevant peptide (HIVp52) or protein (BSA) or bacteria that is not homologous (R. albus). (12B) Total flux (±SEM) of fluorescently labeled T-cells measured in tumors from mice infused with P. aeruginosa-specific or naive T-cells. (12C) Representative fluorescent imaging quantified in 12C. (12D) Mean (±SEM) IL-10 CSPW for tumor infiltrating lymphocytes from mice infused with P. aeruginosa-specific T-cells stimulated with the indicated bacteria. (12E) Mean (±SEM) tumor volume from mice infused with P. aeruginosa-specific (circles) or naive T-cells (triangles). (12F) Mean (±SEM) is shown with horizontal bars for IL-10 CSPW from tumor infiltrating lymphocytes (open circles) or spleen (hatched circles) from C3T mice with spontaneous tumors treated with the indicated bacterial lysate. n=12 mice; **p=0.01. *p<0.05.

FIGS. 13A-13F show that bacterial-tumor antigen cross reactive-specific TORb can be identified in tumor biopsies of newly diagnosed breast cancer patients. Mean (±SD) IL-10 corrected spots per well (CSPW) for PBMC stimulated with BIRC5-p13 or HIF1a-p312 from (13A) Donor 1 and (13B) Donor 2. (13C) Venn diagram for the number of TCRb sequences in the HIF1a-p312-specific T-cell line (left circle) and corresponding tumor biopsy (right circle) from Donor 1. (13D) Percent of the total tumor TCRb repertoire for the indicted TCR for Donor 1. (13E) Venn diagram for the number of TCRb sequences in the BIRC5-p13-specific T-cell line (left circle) and corresponding tumor biopsy (right circle) from Donor 2. (13F) Percent of the total tumor TCRb repertoire for the indicted TCR for Donor 2. *p<0.05.

### DETAILED DESCRIPTION

The invention described herein is based on the discovery and identification of high levels of IL-10 secreting T-cells present in the peripheral blood of both humans (volunteer blood donors or breast cancer patients) and mice, specific for non-mutated tumor antigens (TA), which share significant sequence homologies with bacterial organisms found in the human gut microbiome. These lymphocytes are cross-reactive for both bacterial and tumor antigens. In mice, the bacterial-tumor antigen (BAC-TA) cross-reactive T-cells traffic to tumors and accelerate cancer growth. In this disclosure, we describe use of specific organisms derived from the gut microbiome that we have found rarely or never share homologies with TA, to modulate BAC-TA cross-reactive T-cells. We demonstrate that bacteria with low to no homologies (bacteria^{low}) can be safely enriched in the gut and prevent BAC-TA from expansion. This precision modulation of the gut microbiome reduces tumor growth and increases the efficacy of immune checkpoint inhibitor therapy in a transgenic mouse mammary tumor model of triple negative breast cancer.

Moreover, we have further discovered that use of specific organisms derived from the gut microbiome that share homologies with self-proteins can be used to modulate the autoimmune inflammatory response. This precision modulation of the gut microbiome can be used to elicit a Type 2 immune response.

### DEFINITIONS

All scientific and technical terms used in this application have meanings commonly used in the art unless otherwise specified. As used in this application, the following words or phrases have the meanings specified.

As used herein, the term “comprising” is intended to mean that the compositions and methods include the recited elements, but do not exclude others. As used herein, the transitional phrase “consisting essentially of” (and grammatical variants) is to be interpreted as encompassing the recited materials or steps “and those that do not materially affect the basic and novel characteristic(s)” of the recited embodiment. Thus, the term “consisting essentially of” as used herein should not be interpreted as equivalent to “comprising.” “Consisting of” shall mean excluding more than trace elements of other ingredients and substantial method steps for administering the compositions disclosed herein. Aspects defined by each of these transition terms are within the scope of the disclosure herein.

As used herein, a “linker” refers to an amino acid sequence that connects two proteins, polypeptides, peptides, domains, regions, or motifs and may provide a spacer function compatible with interaction of the two sub-binding domains so that the resulting polypeptide retains a specific binding affinity to a target molecule or retains signaling activity. In certain embodiments, a linker is comprised of about two to about 35 amino acids, for instance, or about four to about 20 amino acids or about eight to about 15 amino acids or about 15 to about 25 amino acids.

As used herein, the terms “nucleic acid sequence” or “polynucleotide” refers to nucleotides of any length which are deoxynucleotides (i.e. DNAs), or derivatives thereof; ribonucleotides (i.e. RNAs) or derivatives thereof; or peptide nucleic acids (PNAs) or derivatives thereof. The terms include, without limitation, single-stranded, double-stranded, or multi-stranded DNA or RNA, genomic DNA, cDNA, DNA-RNA hybrids, oligonucleotides (oligos), or other natural, synthetic, modified, mutated or non-natural forms of DNA or RNA.

The term “vector” refers to, without limitation, a recombinant genetic construct or plasmid or expression construct or expression vector that retains the ability to infect and transduce non-dividing and/or slowly-dividing cells and integrate into the target cell's genome. The vector may be derived from or based on a wild-type virus. Aspects of this disclosure relate to an adeno-associated virus vector, an adenovirus vector, and a lentivirus vector.

The term “expression control element” as used herein refers to any sequence that regulates the expression of a coding sequence, such as a gene. Exemplary expression control elements include but are not limited to promoters, enhancers, microRNAs, post-transcriptional regulatory elements, polyadenylation signal sequences, and introns. Expression control elements may be, without limitation, constitutive, inducible, repressible, or tissue-specific. A “promoter” is a control sequence that is a region of a polynucleotide sequence at which initiation and rate of transcription are controlled. It may contain genetic elements at which regulatory proteins and molecules may bind such as RNA polymerase and other transcription factors. In some embodiments, expression control by a promoter is tissue-specific. An “enhancer” is a region of DNA that can be bound by activating proteins to increase the likelihood or frequency of transcription. Non-limiting exemplary enhancers and posttranscriptional regulatory elements include the CMV enhancer and WPRE.

The term “multicistronic” or “polycistronic” or “bicistronic” or tricistronic” refers to mRNA with multiple, i.e., double or triple coding areas or exons, and as such will have the capability to express from mRNA two or more, or three or more, or four or more, etc., proteins from a single construct. Multicistronic vectors simultaneously express two or more separate proteins from the same mRNA. The two strategies most widely used for constructing multicistronic configurations are through the use of 1) an IRES or 2) a 2A self-cleaving site. An “IRES” refers to an internal ribosome entry site or portion thereof of viral, prokaryotic, or eukaryotic origin which are used within polycistronic vector constructs. In some embodiments, an IRES is an RNA element that allows for translation initiation in a cap-independent manner. The term “self-cleaving peptides” or “sequences encoding self-cleaving peptides” or “2A self-cleaving site” refer to linking sequences which are used within vector constructs to incorporate sites to promote ribosomal skipping and thus to generate two polypeptides from a single promoter, such self-cleaving peptides include without limitation, T2A, and P2A peptides or sequences encoding the self-cleaving peptides.

“Homology” or “identity” or “similarity” refers to sequence similarity between two peptides or between two nucleic acid molecules. Homology can be determined by comparing a position in each sequence which may be aligned for purposes of comparison. When a position in the compared sequence is occupied by the same base or amino acid, then the molecules are homologous at that position. A degree of homology between sequences is a function of the number of matching or homologous positions shared by the sequences. An “unrelated” or “non-homologous” sequence shares less than 40% identity, or alternatively less than 25% identity, with one of the sequences of disclosed herein.

Percent similarity or percent complementary of any of the disclosed sequences may be determined, for example, by comparing sequence information using the GAP computer program, version 6.0, available from the University of Wisconsin Genetics Computer Group (UWGCG). The GAP program utilizes the alignment method of Needleman and Wunsch (1970). Briefly, the GAP program defines similarity as the number of aligned symbols (i.e., nucleotides or amino acids) which are similar, divided by the total number of symbols in the shorter of the two sequences. The preferred default parameters for the GAP program include: (1) a unary comparison matrix (containing a value of 1 for identities and 0 for non-identities) for nucleotides, and the weighted comparison matrix of Gribskov and Burgess (1986), (2) a penalty of 3.0 for each gap and an additional 0.10 penalty for each symbol in each gap; and (3) no penalty for end gaps.

“Nucleotide sequence” refers to a heteropolymer of deoxyribonucleotides, ribonucleotides, or peptide-nucleic acid sequences that may be assembled from smaller fragments, isolated from larger fragments, or chemically synthesized de novo or partially synthesized by combining shorter oligonucleotide linkers, or from a series of oligonucleotides.

As used herein, the terms “protein”, “peptide”, and “polypeptide” refer to amino acid subunits, amino acid analogs, or peptidomimetics. The subunits may be linked by peptide bonds. In another aspect, the subunit may be linked by other bonds, e.g., ester, ether, etc. As used herein the term “amino acid” refers to either natural and/or unnatural or synthetic amino acids.

As used herein, the term “recombinant expression system” or “recombinant expression vector” refers to a genetic construct for the expression of certain genetic material formed by recombination.

The term “effective amount” or “therapeutically effective amount” or “prophylactically effective amount”, refer to an amount of an active agent described herein that is effective to provide the desired/intended result and/or biological activity. Thus, for example, in various embodiments, an effective amount of a composition described herein is an amount that is effective to result in remission or slowing the progression of disease, and/or to improve or to ameliorate symptoms of and/or to treat disease.

When the disclosure herein relates to a small molecule, polypeptide, protein, polynucleotide, nucleic acid, oligonucleotide, antisense, or miRNA, an equivalent or a biologically equivalent of such is intended within the scope of this disclosure. As used herein, the term “biological equivalent thereof” is intended to be synonymous with “equivalent thereof” when referring to a reference small molecule, polypeptide, protein, polynucleotide, nucleic acid, oligonucleotide, antisense, or miRNA even those reference molecules having minimal homology while still maintaining desired structure or functionality. Unless specifically recited herein, it is contemplated that any nucleic acid, polynucleotide, oligonucleotide, antisense, miRNA, polypeptide, or protein mentioned herein also includes equivalents thereof. For example, an equivalent intends at least about 70% homology or identity, or at least 80% homology or identity and alternatively, or at least about 85%, or alternatively at least about 90%, or alternatively at least about 95%, or alternatively 98% percent homology or identity and exhibits substantially equivalent biological activity to the reference protein, polypeptide or nucleic acid. Alternatively, when referring to polynucleotides, an equivalent thereof is a polynucleotide that hybridizes under stringent conditions to the reference polynucleotide or its complement.

In some embodiments disclosed herein, the polypeptide and/or polynucleotide sequences are provided herein for use in gene and protein transfer and expression techniques described herein. Such sequences provided herein can be used to provide the expression product as well as substantially identical sequences that produce a protein that has the same biological properties. These “biologically equivalent” or “biologically active” or “equivalent” polypeptides are encoded by equivalent polynucleotides as described herein. They may possess at least 60%, or alternatively, at least 65%, or alternatively, at least 70%, or alternatively, at least 75%, or alternatively, at least 80%, or alternatively at least 85%, or alternatively at least 90%, or alternatively at least 95% or alternatively at least 98%, identical primary amino acid sequence to the reference polypeptide when compared using sequence identity methods run under default conditions. Specific polynucleotide or polypeptide sequences are provided as examples of particular embodiments. Modifications may be made to the amino acid sequences by using alternate amino acids that have similar charge. Additionally, an equivalent polynucleotide is one that hybridizes under stringent conditions to the reference polynucleotide or its complement or in reference to a polypeptide, a polypeptide encoded by a polynucleotide that hybridizes to the reference encoding polynucleotide under stringent conditions or its complementary strand. Alternatively, an equivalent polypeptide or protein is one that is expressed from an equivalent polynucleotide.

“Hybridization” refers to a reaction in which one or more polynucleotides react to form a complex that is stabilized via hydrogen bonding between the bases of the nucleotide residues. The hydrogen bonding may occur by Watson-Crick base pairing, Hoogstein binding, or in any other sequence-specific manner. The complex may comprise two strands forming a duplex structure, three or more strands forming a multi-stranded complex, a single self-hybridizing strand, or any combination of these. A hybridization reaction may constitute a step in a more extensive process, such as the initiation of a PC reaction, or the enzymatic cleavage of a polynucleotide by a ribozyme.

Examples of stringent hybridization conditions include: incubation temperatures of about 25 °C to about 37 °C; hybridization buffer concentrations of about 6×SSC to about 10×SSC; formamide concentrations of about 0% to about 25%; and wash solutions from about 4×SSC to about 8×SSC. Examples of moderate hybridization conditions include: incubation temperatures of about 40 °C to about 50 °C; buffer concentrations of about 9×SSC to about 2×SSC; formamide concentrations of about 30% to about 50%; and wash solutions of about 5×SSC to about 2×SSC. Examples of high stringency conditions include: incubation temperatures of about 55 °C to about 68 °C; buffer concentrations of about Ix SSC to about 0.1×SSC; formamide concentrations of about 55% to about 75%; and wash solutions of about Ix SSC, 0.1×SSC, or deionized water. In general, hybridization incubation times are from 5 minutes to 24 hours, with 1, 2, or more washing steps, and wash incubation times are about 1, 2, or 15 minutes. SSC is 0.15 M NaCl and 15 mM citrate buffer. It is understood that equivalents of SSC using other buffer systems can be employed.

As used herein, “treating” or “treatment” of a disease in a subject refers to (1) preventing the symptoms or disease from occurring in a subject that is predisposed or does not yet display symptoms of the disease; (2) inhibiting the disease or arresting its development; or (3) ameliorating or causing regression of the disease or the symptoms of the disease. As understood in the art, “treatment” is an approach for obtaining beneficial or desired results, including clinical results.

As used herein, the term “isolated” means that a naturally occurring DNA fragment, DNA molecule, coding sequence, or oligonucleotide is removed from its natural environment, or is a synthetic molecule or cloned product. Preferably, the DNA fragment, DNA molecule, coding sequence, or oligonucleotide is purified, i.e., essentially free from any other DNA fragment, DNA molecule, coding sequence, or oligonucleotide and associated cellular products or other impurities.

The term “cell” as used herein refers to either a prokaryotic or eukaryotic cell, optionally obtained from a subject or a commercially available source. Cells treated, transfected, transformed, or otherwise in contact with compositions and/or nucleic acid molecules disclosed herein, include without limitation, cells of a human, non-human animal, mammal, or non-human mammal, including without limitation, cells of murine, canine, or non-human primate species. Cells treated, transfected, transformed, or otherwise in contact with compositions and/or nucleic acid molecules disclosed herein are, without limitation, T cells, antigen-presenting cells, and other suitable host cells.

As used herein, the term “subject” includes any human or non-human animal. The term “non-human animal” includes all vertebrates, e.g., mammals and non-mammals, such as non-human primates, horses, sheep, dogs, cows, pigs, chickens, and other veterinary subjects.

As used herein, “a” or “an” means at least one, unless clearly indicated otherwise.

As used herein, to “prevent” or “protect against” a condition or disease means to hinder, reduce or delay the onset or progression of the condition or disease.

The term “encode” as it is applied to nucleic acid sequences refers to a polynucleotide which is said to “encode” a polypeptide, an mRNA, or an effector RNA if, in its native state or when manipulated by methods well known to those skilled in the art, can be transcribed and/or translated to produce the effector RNA, the mRNA, or an mRNA that can for the polypeptide and/or a fragment thereof. The antisense strand is the complement of such a nucleic acid, and the encoding sequence can be deduced therefrom.

As used herein, the term “expression” or “gene expression” refers to the process by which polynucleotides are transcribed into mRNA and/or the process by which the transcribed mRNA is subsequently translated into peptides, polypeptides, or proteins. If the polynucleotide is derived from genomic DNA, expression may include splicing of the mRNA in a eukaryotic cell. The expression level of a gene may be determined by measuring the amount of mRNA or protein in a cell or tissue sample; further, the expression level of multiple genes can be determined to establish an expression profile for a particular sample.

As used herein, the term “functional” may be used to modify any molecule, biological, or cellular material to intend that it accomplishes a particular, specified effect.

The term “about,” as used herein when referring to a measurable value such as an amount, level or concentration, for example and without limitation, is meant to encompass variations of 20%, 10%, 5%, 1%, 0.5%, or even 0.1% of the specified amount, or fold differences in levels of a quantifiable comparison with a standard or control or reference material, such as 1-fold, 2-fold, 3-fold, 4-fold . . . 10-fold, 100-fold, etc. of the specified level of comparison.

The terms “acceptable,” “effective,” or “sufficient” when used to describe the selection of any components, ranges, dose forms, etc. disclosed herein intend that said component, range, dose form, etc. is suitable for the disclosed purpose.

### ABBREVIATIONS

BAC-TA: bacterial tumor antigen
bacteria^{high}: bacteria in the gut microbiome with many (greater than 50%) homologies to tumor antigens
bacteria^{low}: bacteria in the gut microbiome with few to no (less than 50%) homologies to tumor antigens
BSA: bovine serum albumin
C3T: TgC3(1)-Tag transgenic mouse
CFU: colony forming units
CSPW: corrected spots/well
FNA: fine needle aspiration
IFN-g: interferon-gamma
IL: interleukin
IVS: in vitro stimulation
MDSC: myeloid derived suppressor cells
mLN: mesenteric lymph nodes
PBMC: peripheral blood mononuclear cells
PRL-3: Phosphatase of regenerating liver 3
SPF: specific pathogen free
TA: tumor antigen
TCR: T-cell receptor
Th1: T-helper 1 T-cell
Th2: T-helper 2 T-cell
TIL: tumor infiltrating lymphocytes
YB-1: Y-box binding protein

### Methods & Bacterial Compositions for Treating Cancer

Described herein are methods for enhancing immunity to a tumor antigen in a subject, as well as for slowing tumor growth in a subject in need thereof. In some embodiments, these methods comprise administering to the subject an effective amount of a composition comprising a plurality of bacteria that exhibit less than 50% amino acid sequence homology to non-mutated tumor antigens (bacteria^{low}). In some embodiments, the plurality of bacteria^{low} are selected from: Lactobacillus acidophilus, Ruminococcus albus, Ruminococcus gnavus, Dorea formicigenerans, Eubacterium siraeum, Bacteroides stercoris, Parabacteroides distasonis, Bifidobacterium breve, Lactobacillus delbrueckii, Odoribacter splanchnicus, Akkermansia muciniphila, Desulfovibrio piger, Mitsuokella multacida, Bifidobacterium catenulatum, Staphylococcus saprophyticus, Bacteroides vulgatus, Bifidobacterium pseudocatenulatum, Bifidobacterium angulatum, Bifidobacterium bifidum, Campylobacter hominis, Clostridium leptum, Clostridium methylpentosum, Coprococcus catus, Eubacterium ventriosum, Ruminococcus bromii, Victivallis vadensis, Bacteroides eggerthii, Bacteroides helcogenes, Granulicatella adiacens, Peptoniphilus harei In some embodiments, the plurality of bacteria^{low} comprise at least four bacteria selected from: R. albus, L. acidophilus, A. muciniphila, B. breve, O. splanchnicus and P. distasonis. In some embodiments, the plurality of bacteria^{low} comprise R. albus and L. acidophilus.

TABLE 1
Bacteria Relevant to Cancer

| | |
|---|---|
| Lactobacillus acidophilus | Bifidobacterium angulatum |
| Ruminococcus albus | Bifidobacterium bifidum |
| Ruminococcus gnavus | Campylobacter hominis |
| Dorea formicigenerans | Clostridium leptum |
| Eubacterium siraeum | Clostridium methylpentosum |
| Bacteroides stercoris | Coprococcus catus |
| Parabacteroides distasonis | Eubacterium ventriosum |
| Bifidobacterium breve | Ruminococcus bromii |
| Lactobacillus delbrueckii | Victivallis vadensis |
| Odoribacter splanchnicus | Bacteroides eggerthil |
| Akkermansia muciniphila | Bacteroides helcogenes |
| Desulfovibrio piger | Granulicatella adiacens |
| Mitsuokella multacida | Peptoniphilus harei |
| Bifidobacterium catenulatum | Bifidobacterium angulatum |
| Staphylococcus aprophyticus | Bifidobacterium bifidum |
| Bacteroides vulgatus | Campylobacter hominis |
| Bifidobacterium pseudocatenulatum | Clostridium leptum |

In some embodiments, the composition is free of bacteria exhibiting greater than 50% homology to tumor antigens identified in a blood sample obtained from the subject. In some embodiments, the method further comprises obtaining such a blood sample from the subject, and identifying bacterial-tumor antigen (BAC-TA) cross-reactive memory T-cells detected in the sample. In some embodiments, the composition is administered weekly for six weeks. In some embodiments, the composition is administered twice a week. In some embodiments, the composition is administered twice a month. In some embodiments, the composition is administered monthly. In some embodiments, the administration continues for at least two months. In some embodiments, the administration continues for at least 6, 8, 12, or 24 months.

In some embodiments, the effective amount is sufficient to effect a two-fold increase in gut microbiome bacteria^{low}, and/or a significant decrease in bacterial-tumor antigen (BAC-TA) cross-reactive memory T-cells, in a biological sample obtained from the subject. In some embodiments, the method enhances efficacy of a cancer treatment. In some embodiments, the cancer treatment comprises immunotherapy and/or chemotherapy. In some embodiments, the immunotherapy comprises administration of immune checkpoint inhibitor therapy, vaccine therapy, cytokine therapy, and/or oncolytic virus therapy. In some embodiments, the chemotherapy comprises administration of paclitaxel. Other chemotherapeutic agents, as well as other immunotherapeutic agents, known to those skilled in the art are likewise contemplated. Some representative examples of chemotherapeutic agents include, but are not limited to, cisplatin, carboplatin, pemtrexed, Adriamycin, Cytoxan. Some representative examples of immunotherapeutic agents include, but are not limited to, monoclonal antibodies, such as, for example, anti-HER2, anti-EGFR, and anti-VEGF. In some embodiments, the composition is administered prior to and/or concomitantly with the immunotherapy or chemotherapy.

In some embodiments, the method enhances a type 1 immune response. In some embodiments, the method increases the relative amount of bacteria^{low} that can be detected in a biological sample obtained from the subject. Examples of biological samples include blood and stool samples.

In some embodiments, the composition comprises 1-100×10⁹ CFU of bacteria^{low}. In some embodiments, the composition comprises 1-50×10⁹ CFU of bacteria^{low}. In some embodiments, the composition comprises 1-10×10⁹ CFU of bacteria^{low}. In some embodiments, the composition comprises 1-5×10⁹ CFU of bacteria^{low}.

In some embodiments, the administration of the composition is oral, rectal, or intragastric. In some embodiments, the composition is delivered in the form of a suppository.

Also described is a composition comprising a plurality of bacteria that exhibit less than 50% homology to non-mutated tumor antigens (bacteria^{low}). In some embodiments, the plurality of bacteria^{low} are selected from: Lactobacillus acidophilus, Ruminococcus albus, Ruminococcus gnavus, Dorea formicigenerans, Eubacterium siraeum, Bacteroides stercoris, Parabacteroides distasonis, Bifidobacterium breve, Lactobacillus delbrueckii, Odoribacter splanchnicus, Akkermansia muciniphila, Desulfovibrio piger, Mitsuokella multacida, Bifidobacterium catenulatum, Staphylococcus saprophyticus, Bacteroides vulgatus, Bifidobacterium pseudocatenulatum, Bifidobacterium angulatum, Bifidobacterium bifidum, Campylobacter hominis, Clostridium leptum, Clostridium methylpentosum, Coprococcus catus, Eubacterium ventriosum, Ruminococcus bromii, Victivallis vadensis, Bacteroides eggerthii, Bacteroides helcogenes, Granulicatella adiacens, Peptoniphilus harei In some embodiments, the plurality of bacteria^{low} comprise at least four bacteria selected from: R. albus, L. acidophilus, A. muciniphila, B. breve, O. splanchnicus and P. distasonis. In some embodiments, the plurality of bacteria^{low} comprise R. albus and L. acidophilus. In some embodiments, the composition is substantially free of bacteria that exhibit greater than 50% homology to tumor antigens.

### Methods & Bacterial Compositions for Treating Autoimmune Inflammatory Responses

A method of ameliorating an autoimmune inflammatory response in a subject is also provided. In some embodiments, the method comprises administering to the subject an effective amount of a composition comprising a plurality of bacteria that exhibit at least 50% (high) homology to self-proteins (bacteria^{high}). In some embodiments, the plurality of bacteria^{high} are selected from: Escherichia coli, Enterococcus faecalis, Pseudomonas fluorescens, Pseudomonas putida, Pseudomonas aeruginosa, Shigella dysenteriae, Enterobacter cloacae, Lactococcus lactis, Clostridium perfringens, Klebsiella pneumoniae, Staphylococcus aureus, Enterococcus faecium, Rhodopseudomonas palustris, Shigella boydii, Shigella sonnei, Finegoldia magna, Clostridium asparagiforme, Collinsella aerofaciens, Fusobacterium ulcerans, Aerococcus viridans, Bacteroides ovatus, Clostridium botulinum, Enterococcus gallinarum, Eubacterium limosum, Faecalibacterium prausnitzii, Holdemania filiformis, Lactobacillus plantarum, Lactobacillus reuteri, Marvinbryantia formatexigens, Megamonas hypermegale, Streptococcus agalactiae, Streptococcus anginosus, Streptococcus dysgalactiae, and Streptococcus infantarius. In some embodiments, the plurality of bacteria^{high} exhibit at least 50% homology to self-proteins identified in a blood and/or stool sample obtained from the subject.

TABEL 2
Bacteria Relevant to Autoimmunity/Inflammatory Conditions

| | |
|---|---|
| Escherichia coli | Collinsella aerofaciens |
| Enterococcus faecalis | Fusobacterium ulcerans |
| Pseudomonas fluorescens | Aerococcus viridans |
| Pseudomonas putida | Bacteroides ovatus |
| Pseudomonas aeruginosa | Clostridium botulinum |
| Shigella dysenteriae | Enterococcus gallinarum |
| Enterobacter cloacae | Eubacterium limosum |
| Lactococcus lactis | Faecalibacterium prausnitzii |
| Clostridium perfringens | Holdemania filiformis |
| Klebsiella pneumoniae | Lactobacillus plantarum |
| Staphylococcus aureus | Lactobacillus reuteri |
| Enterococcus faecium | Marvinbryantia formatexigens |
| Rhodopseudomonas palustris | Megamonas hypermegale |
| Shigella boydii | Streptococcus agalactiae |
| Shigella sonnei | Streptococcus anginosus |
| Finegoldia magna | Streptococcus dysgalactiae |
| Clostridium asparagiforme | Streptococcus infantarius |

In some embodiments, the composition is administered weekly for six weeks to one year. In some embodiments, the composition is administered weekly for at least one year. In some embodiments, the composition is administered twice a week. In some embodiments, the composition is administered twice a month. In some embodiments, the composition is administered monthly. In some embodiments, the administration continues for at least two months. In some embodiments, the administration continues for at least 6, 8, 12, or 24 months.

In some embodiments, the effective amount is sufficient to effect a two-fold increase in gut microbiome bacteria^{high} in a biological sample obtained from the subject. Examples of biological samples include blood and stool samples. In some embodiments, the method further comprises obtaining a biological sample from the subject, and identifying self-protein reactive memory T-cells.

In some embodiments, the method elicits a type 2 immune response. In some embodiments, the method elicits an increase in the amount of IL-10 and/or IL-6 that can be detected in a biological sample obtained from the subject.

In some embodiments, the composition 1-100×10⁹ CFU of bacteria^{high} In some embodiments, the composition comprises 1-50×10⁹ CFU of bacteria^{high}. In some embodiments, the composition comprises 1-10×10⁹ CFU of bacteria^{high} In some embodiments, the composition comprises 1-5×10⁸ CFU of bacteria^{high}.

In some embodiments, the administration of the composition is oral, rectal, or intragastric. In some embodiments, the composition is delivered in the form of a suppository.

Also described is a composition comprising a plurality of bacteria that exhibit at least 50% amino acid sequence homology to self-proteins (bacteria^{high}). In some embodiments, the plurality of bacteria^{high} are selected from: Escherichia coli, Enterococcus faecalis, Pseudomonas fluorescens, Pseudomonas putida, Pseudomonas aeruginosa, Shigella dysenteriae, Enterobacter cloacae, Lactococcus lactis, Clostridium perfringens, Klebsiella pneumoniae, Staphylococcus aureus, Enterococcus faecium, Rhodopseudomonas palustris, Shigella boydii, Shigella sonnei, Finegoldia magna, Clostridium asparagiforme, Collinsella aerofaciens, Fusobacterium ulcerans, Aerococcus viridans, Bacteroides ovatus, Clostridium botulinum, Enterococcus gallinarum, Eubacterium limosum, Faecalibacterium prausnitzii, Holdemania filiformis, Lactobacillus plantarum, Lactobacillus reuteri, Marvinbryantia formatexigens, Megamonas hypermegale, Streptococcus agalactiae, Streptococcus anginosus, Streptococcus dysgalactiae, and Streptococcus infantarius.

In some embodiments, the plurality comprises at least four species of bacteria^{high}. In some embodiments, the plurality comprises 6-10 species of bacteria^{high}.

### Pharmaceutical Compositions, Administration, and Dosage

In some embodiments, the subject to be treated in the above methods is human. In some embodiments, the subject is suspected of having, or has been diagnosed with cancer, such as breast cancer, or an autoimmune disease. Determination of amounts and appropriate means of administration are determined under the guidance of a treating physician, taking into account an individual patient's condition.

Pharmaceutical compositions disclosed herein include one or more pharmaceutically or physiologically acceptable carriers, diluents or excipients. Such compositions may comprise buffers such as neutral buffered saline, phosphate buffered saline and the like; carbohydrates such as glucose, mannose, sucrose or dextrans, mannitol; proteins; peptides; adjuvants (e.g., aluminum hydroxide); and preservatives.

The compositions and/or bacteria disclosed herein are administered in any suitable manner, often with pharmaceutically acceptable carriers. Suitable methods of administering compositions in the context of the present invention to a subject are available, and, although more than one route can be used to administer a particular composition, a particular route can often provide a more immediate and more effective reaction than another route. Compositions of the disclosure may be formulated for administration as a suppository, or for oral delivery.

The dose administered to a patient, in the context of the disclosure herein, should be sufficient to result in a beneficial therapeutic response in the patient over time, or to inhibit disease progression. Thus, the composition is administered to a subject in an amount sufficient to elicit an effective response and/or to alleviate, reduce, cure or at least partially arrest symptoms and/or complications from the disease. An amount adequate to accomplish this is defined as a “therapeutically effective dose.”

Routes, order and/or frequency of administration of the therapeutic compositions disclosed herein, as well as dosage, will vary from individual to individual, and may be readily established using standard techniques. In general, an appropriate dosage and treatment regimen provides the active compound(s) in an amount sufficient to provide therapeutic and/or prophylactic benefit. Such a response can be monitored by establishing an improved clinical outcome in treated patients as compared to non-treated patients.

### EXAMPLES

The following examples are presented to illustrate the present invention and to assist one of ordinary skill in making and using the same. The examples are not intended in any way to otherwise limit the scope of the invention.

### Example 1: BAC-TA Cross-Reactive Memory T-Cells are Present in the Peripheral Blood of Volunteer Donors and Breast Cancer Patients

We developed a method of CD4 T-cell epitope identification that includes identifying putative Class II interacting sequences via a multi-algorithm approach to ensure responsiveness across diverse HLA DR alleles followed by functional phenotyping of the Th response (3). Using these methods, we have subsequently evaluated 152 epitopes from 17 non-mutated tumor antigens and demonstrated that Class II restricted epitopes could be identified from each protein which preferentially elicit either a selective Th1 (IFN-g) or Th2 (IL-10) response across multiple volunteer or cancer donors (n=40). The resultant database has allowed us to explore characteristics that may define CD4 Th1 vs. Th2 selective epitopes. We have found no significant association with the cellular location of the epitope nor the number of commonly reported mutations within 10 amino acids of an epitope and either a Th1 or Th2 response generated by that epitope. There are no specific amino acid motifs that are associated with either phenotype.

When we evaluated sequence homologies for all epitopes, however, we found that there was a significant association of numerous amino acid sequence homologies to bacterial species found in the gut microbiome with the IL-10 inducing TA peptides. Tumor antigen epitopes most likely to be IL-10 inducing shared a >50% identity with multiple gut bacterial species, in some cases over 20 species. The higher the magnitude IL-10 response elicited by a Th2 selective TA epitope in either a volunteer or breast cancer donor, the greater the number of bacterial homologies associated with that epitope (p<0.0005). No association occurred between IFN-g magnitude and number of bacterial amino acid alignments. We have been able to identify a selected number of bacterial species that are most commonly associated with numerous TA homologies which we call bacteria^{high} We have also identified bacteria in the gut microbiome that are rarely or never found to be homologous to any tumor antigen sequences, which we call bacteria^{low}.

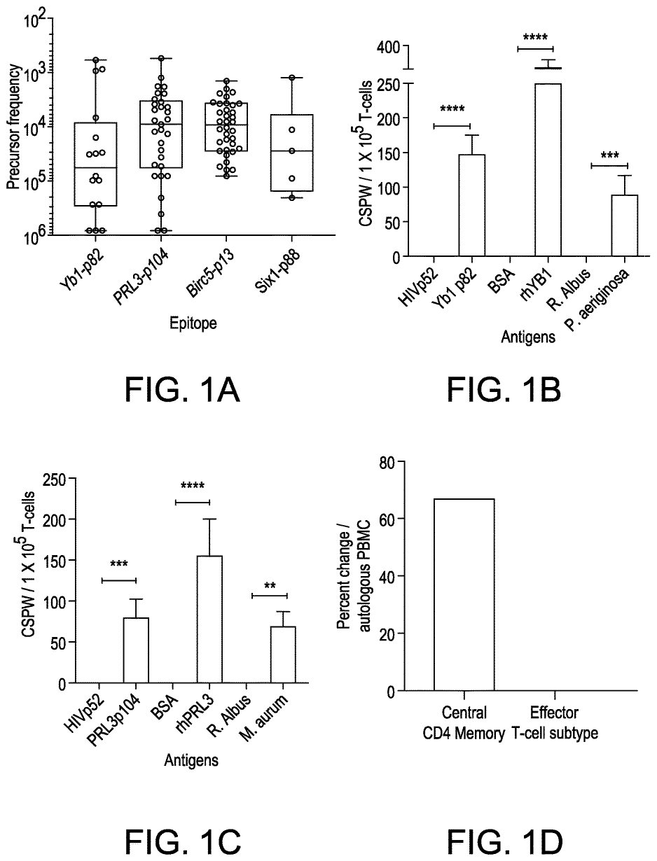

BAC-TA cross-reactive T-cells specific for bacteria^{high} are readily identified in the peripheral blood of both volunteer donors and breast cancer patients. Thirty-six percent (14/40) of donors tested demonstrated increased circulating IL-10 secreting T-cells specific for a Y-box binding protein (YB1)-82-96 epitope with a median level of 1:50,000 (range, 1:600-1:96K) cells in peripheral blood mononuclear cells (PBMC) (FIG. 1A). Phosphatase of regenerating liver 3 (PRL3)-p104-118 epitope specific IL-10 secreting T-cells were observed in 69% of donors with a median level of 1:9,000 (range, 1:2K-1:80K) of PBMC (FIG. 1A). Eighty percent of donors had 1:9,000 (range, 1:1K-1:80K) PBMC specific for survivin (BIRC5)-p13-27 (FIG. 1A). Eleven percent of donors demonstrated increased circulating homeobox protein Six1 (Six1)-p88-102 epitope specific IL-10 secreting T-cells with a median level of 1:28,000 (range, 1:1K-1:88K) in PBMC. These studies were performed with a direct IL-10 ELISPOT.

We demonstrate that BAC-TA cross-reactive epitope-specific T-cell lines not only recognize the corresponding human recombinant oncoprotein, but also secrete IL-10 in response to the bacteria^{high} that shares greater than 50% homology to that peptide. YB1-p82-96-specific T-cells secreted significantly more IL-10 when incubated with a homologous bacteria^{high}, P. aeruginosa, compared to a non-homologous bacteria^{low}, R. albus (p=0.001; FIG. 1B). Similarly, there was significantly more IL-10 secretion observed when the PRL3-p104-118-specific T-cell line was stimulated with the homologous bacteria^{high}, M. aurum, compared to a non-homologous bacteria^{low}, R. albus (p=0.005; FIG. 1C). T-cell responses were class II restricted for the peptide, bacterial, and recombinant oncogenic protein antigens. Flow cytometric analysis of the antigen-specific T-cell lines demonstrated an enrichment of central memory T-cells when compared to the autologous PBMC (FIG. 1D).

### Example 2: BAC-TA T-Cells can Traffic to Tumor and Accelerate Mammary Cancer Growth

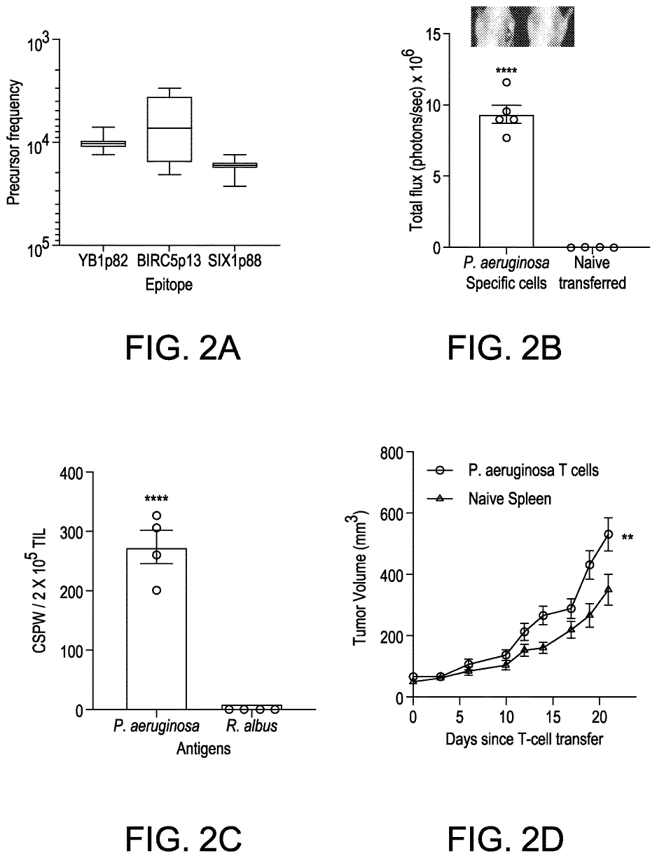

BAC-TA cross-reactive T-cells can also be identified in the spleens of mice (FVB/n strain). The median level of circulating YB1-p82 specific IL-10 secreting T-cells was 1:10,000 (range 1:10,000-1:71,000) of splenocytes from 30% of murine donors. Sixty percent of the mice responded to BIRC5-p13 by secreting IL-10 at a median precursor frequency of 1:7000 (range 1:4000-1:76,000). The median precursor frequency of Six1-p88 specific IL-10 secreting T-cells was 1:17,000 (range 1:13,000-1:20,000) splenocytes from 30% of donors (FIG. 2A). We generated P. aeruginosa-specific T-cells from a specific-pathogen free (SPF) naïve FVB mouse and demonstrated the P. aeruginosa-specific T-cell line secreted significantly more IL-10 when stimulated with the homologous peptide YB1-p82-96 (70% homologous) as compared to a non-homologous peptide (HIV-p52-66; p=0.0004) and to the recombinant human YB1 protein as compared to an irrelevant protein, BSA (p<0.0001).

We implanted a syngeneic tumor cell line (M6) into the TgC3 (1)-Tag (C3T) transgenic mouse mammary fat pad. The C3T genetically engineered mouse model of mammary cancer is a model of “basal like” triple negative breast cancer. Four weeks later, when the tumors measured 65 (±32) mm³, 10×10⁶ P. aeruginosa-YB-1 BAC-TA cross-reactive T-cells or naïve splenocytes that had been fluorescently-labeled with XenoLight DIR (Perkin Elmer) were infused intravenously. After 72 hours, we observed that 100% of the mice injected with fluorescently-labeled P. aeruginosa-YB1 BAC-TA cross-reactive T-cells demonstrated significantly increased florescence in the tumors, whereas no fluorescence was observed in the mice injected with labeled naïve splenocytes (p<0.0001; FIG. 2B). Representative pictures of T-cell infused mice from each group are shown in FIG. 2B. We confirmed that the tumor infiltrating lymphocytes (TIL) were specific for P. aeruginosa. Indeed, significantly higher magnitude IL-10 secreting cells were observed from TIL stimulated with P. aeruginosa (272±28 cSPW) as compared to R. albus (0 cSPW; p<0.0001; FIG. 2C). We further demonstrated that P. aeruginosa-YB1 cross-reactive T-cells promoted tumor growth. In a repeat experiment with unlabeled cells, tumor volume measured 20 days after T-cell transfer was increased by 37% in the mice who received the BAC-TA cross-reactive T-cells as compared to those injected with naïve splenocytes (p=0.01; FIG. 2D).

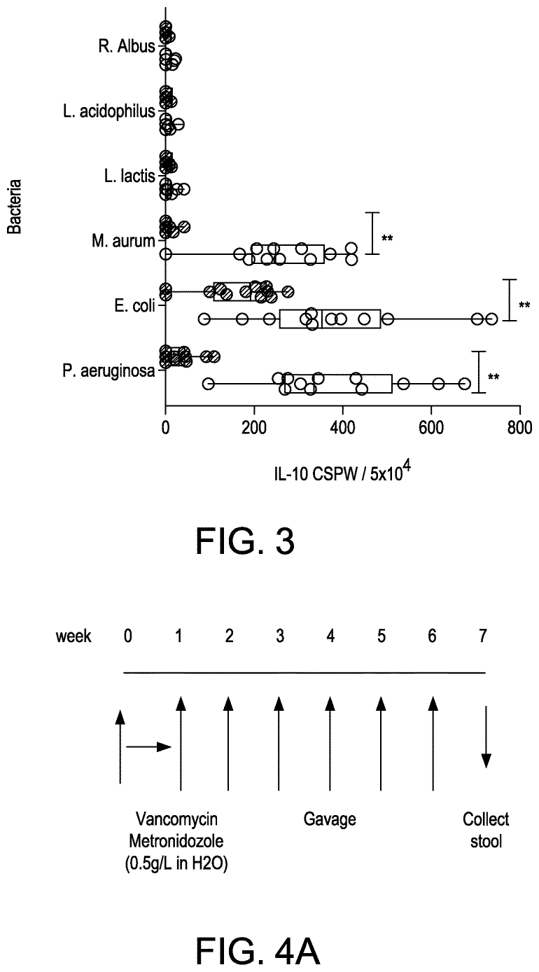

BAC-TA cross-reactive T-cells are found in spontaneous mammary tumors developing in the C3T mouse. We allowed mice to develop spontaneous tumors to a size of ~500±75 mm3. The tumors were then excised and enzymatically dissociated using the mouse TDK kit in combination with gentleMACs (Miltenyi Biotec). TIL were isolated via gradient centrifugation. In an ELISPOT analysis, TIL were enriched for IL-10-secreting T-cells specific for bacteria that share >50% homology with Th2-selective epitopes from tumor associated antigens expressed in the C3T spontaneous tumors (P. aeruginosa/70% homology with YB1 epitope, E. coli/70% homology with YB1 epitope, and M. aurum/80% homology with PRL3 epitope) as compared to the bacterial specific T-cell levels in spleen (p<0.01 for all). As controls, we tested three bacteria that do not share any homology with the YB1 or PRL3, L. lactis, L. acidophilus and R. albus. These non-homologous bacteria did not induce IL-10 secretion in the isolated TIL (FIG. 3). These data suggest BAC-TA cross-reactive T-cells can be detrimental if an individual develops breast cancer. The T-cells have been primed for antigen in the gut and may rapidly expand when the homologous tumor antigen is upregulated in breast cancer.

### Example 3: Bacteria^{low} can be Enriched in the Gut Microbiome and Augment Immunity to a Vaccine Targeting the Non-Mutated Tumor Antigen IGFBP-2

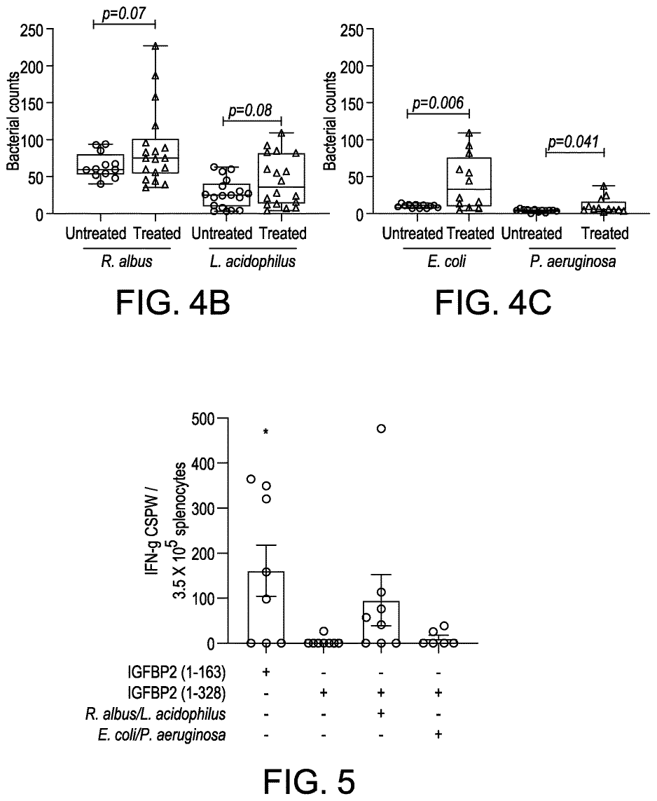

SPF TgMMTV-neu mice, a transgenic model of luminal B murine mammary cancer, were treated for one week with vancomycin and metronidazole (0.5 mg/kg) in the drinking water. This protocol reduces high frequency species in the gut by >90% as assayed via 16s rRNA sequencing. The mice received weekly (for 6 weeks) oral treatments of 1×10⁹ CFU of a combination of L. acidophilus and R. albus, both bacteria^{low} species or a combination of E. coli and P. aeruginosa, both bacteria^{high} species. Stool was collected 1 week after the last dose of bacteria and underwent metagenomic sequencing (FIG. 4A). After treatment with the pool of L. acidophilus and R. albus, there was a 28% increase in R. albus (p=0.07) and a 38% increase in L. acidophilus (p=0.08) in the stool as compared to the bacterial counts detected in the untreated stool (FIG. 4B). When mice were treated with E. coli/P. aeruginosa, we observed a 65% increase in E. coli (p=0.006) and a 32% increase in P. aeruginosa (p=0.041) in the stool as compared to the control (FIG. 4C).

Using the same protocol as is described in FIG. 4, we fed mice with L. acidophilus/R. albus and then implanted the syngeneic breast cancer cell line (MMC; 0.5×10⁶; subcutaneously) two weeks after the last bacteria dose. At study termination, treatment with L. acidophilus/R. albus resulted in a significantly reduced tumor volume (mean, 345±42 mm³) as compared to untreated mice (mean, 544±71 mm³; p<0.0001).

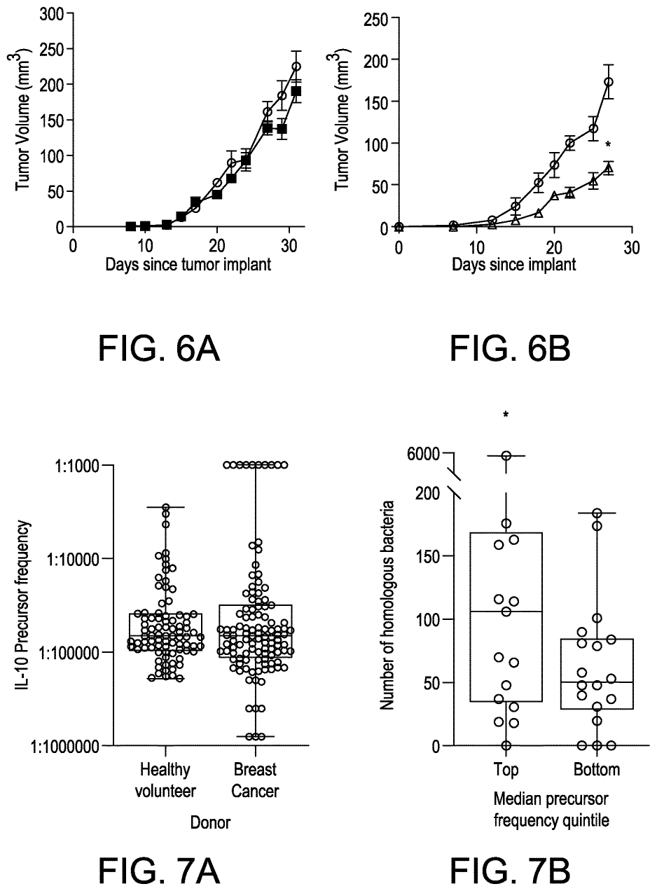

We have previously published the functional Th phenotype for the IGFBP-2 protein (3). The majority of the IFN-g inducing Th1 selective epitopes are found in the N-terminus of the protein (aa 1-163). A vaccine consisting of the N-terminus could inhibit tumor growth. The C-terminus of IGFBP-2, in contrast, was enriched with Th2 selective epitopes secreting high levels of IL-10. A C-terminus vaccine (aa 164-328), when used for immunization, had no effect on tumor growth. Moreover, if the N-terminus and C-terminus sequences were combined, the anti-tumor effect of N-terminus immunization was completely abrogated showing the immunologic dominance of the IL-10 inducing epitopes (3). We questioned whether enriching the microbiome with bacteria^{low} might enhance the immunogenicity of the C-terminus or the whole protein vaccine presumably by reducing antigenic stimulation to BAC-TA cross-reactive T-cells. Using the same protocol as is described in FIG. 4, we fed mice with L. acidophilus/R. albus and then vaccinated with the N-terminus vaccine or the full length IGFBP-2 protein encoded in DNA (3 vaccines over 6 weeks). Two weeks after the last vaccine, antigen specific immunity was measured by IFN-g ELISPOT (FIG. 5). As expected, immunization with IGFBP-2 (1-163, N-terminus) in untreated mice induced a Th1 response and immunization with full-length IGFBP-2 demonstrated no IFN-g response in control mice (p=0.012; FIG. 5). However, after mice were treated with L. acidophilus/R. albus, the full-length vaccine significantly induced IFN-g secretion (95±59 CSPW; p=0.022; FIG. 5). Feeding with bacteria^{high} did not induce Type I immune responses.

These data provide evidence that manipulation of the gut microbiome to enrich for bacteria^{low} species confers beneficial effects on the ability to elicit Type I immune responses.

### Example 4: Precision Modulation of the Gut Microbiome with Bacteria^{low} can Limit Tumor Growth in a Transgenic Mammary Tumor Model of Triple Negative Breast Cancer and Improve Response to Immunotherapy

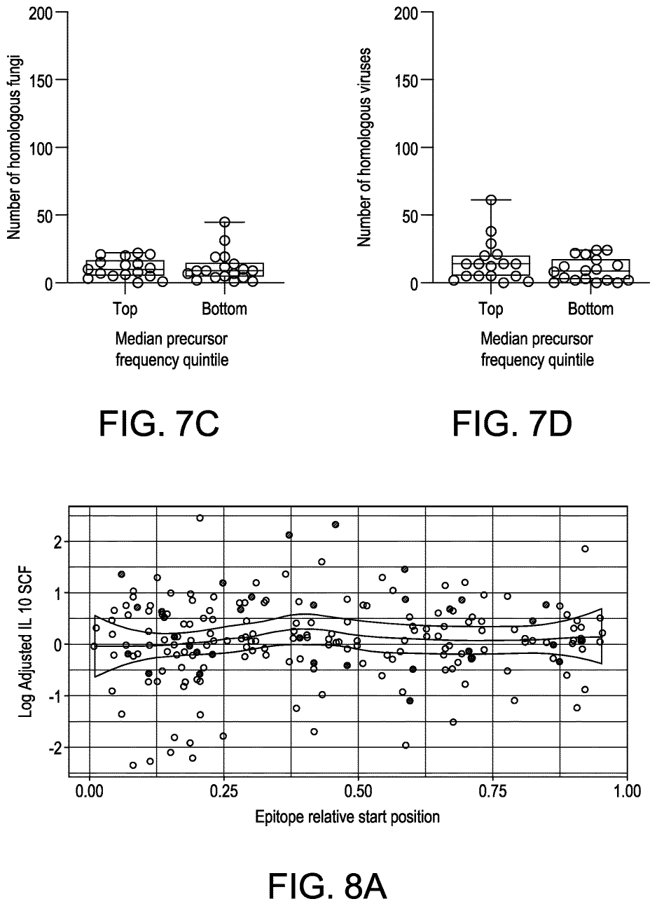

We utilized the C3T genetically engineered mouse model of mammary cancer and a syngeneic tumor cell line derived from a spontaneous tumor from these mice, M6. The C3T reflects a basal triple negative breast cancer phenotype, tumors express PD-L1, and the model is in an FVB/n background. Thus assays and microbiome data are relevant to this model. We have sequenced both the spontaneous tumors in these animals as well as the M6 cell line. Both are highly enriched for nonsynonymous mutations which could allow the development of spontaneous immune responses to neoantigens. Moreover, the C3T has an aggressive tumor that is resistant to both immune and chemotherapy (FIG. 6). C3T mice were implanted with the syngeneic basal breast cancer cell line (M6) then treated i.p. with anti-PD-L1 antibody (200 ug) three days after implantation then twice weekly until the termination of the study (FIG. 6A). Paclitaxel therapy was given i.v. at 10 mg/kg one day after implantation and 3 times per week until the termination of the study (FIG. 6B). Tumor volumes in the C3T after treatment with anti-PD-L1 were no different than in untreated mice (p=0.91; FIG. 6A). Although paclitaxel could moderately inhibit the growth of M6 as compared to control (p=0.048; FIG. 6B), tumors continued to grow. We evaluated microbiome manipulation to slow progression of spontaneous disease and show that gut microbiome enrichment with bacteria^{low} can improve clinical response to immune checkpoint inhibitor therapy via increased generation of endogenous Type I T-cells in tumors.

We show that treatment with bacteria^{low} can limit tumor growth in C3T spontaneous mammary tumor model. We have characterized the natural history of spontaneous mammary tumor growth in the C3T mice as well as the baseline phenotype of TIL in untreated animals. C3T develop tumors at a mean age of 18.5±2.83 weeks and the TIL express predominantly Gata 3, a marker of Th2 (12). Mice were treated with bacteria starting at 6 weeks of age or sham gavage at which time the animals were observed for tumor development two to three times per week until sacrifice. A fine needle aspiration (FNA) was taken every two weeks once tumors arose to monitor changes in the immune infiltrate and TCRVb consistent with BAC-TA T-cells over time. We have extensive experience performing FNA on mouse tumors and studies from our animal core have shown serial FNA do not significantly alter the ability to compare tumor volumes to control. Tumor volumes were calculated from raw measurements by the standard volume calculation for an ellipsoid (12). Tumor growth kinetics were calculated by determining the change in volume between subsequent measurements and dividing by the number of days between the measurements, resulting in a rate value of mm³/day. We sacrificed the cohorts when tumors from all the control mice had reached 1000±200 mm³. We found that a greater percentage of mice in the control group will have palpable tumors at week 24, which is the peak of tumor development in this model, than mice in the bacteria^{low} fed group.

To determine the phenotype of TIL and additional immune infiltrates, we performed flow cytometric analysis and TCRVb sequencing to assess clonality using the FNA samples. An initial assessment of the T-cell clonality of the tumor includes the evaluation of the Shannon index, which is a quantitative measure that reflects diversity. Lower values indicate more diversity while higher values indicate less diversity, which would suggest the development of specific T-cell clones in the tumor.

To determine whether treatment with bacterial can enhance the response to an immune checkpoint inhibitor in the M6 implant model. Mice will be treated with bacteria in the optimal regimen or sham control as defined above. Based on results of the experiments above that the changes in gut microbiome persist for some time after feeding, we will administer probiotics prior to tumor implant. If we find that the bacterial changes are not persistent, we can time our oral therapy with treatment so that we know the gut microbiome of the mice is enriched with bacteria^{low} through the experiment. Mice will be implanted with the syngeneic tumor cell line (M6) in a manner similar to that described in FIGS. 6 and 24 hours later, animals will be treated with an anti-PD-L1 antibody (200 ug/mouse, injected i.p. every other day for a total of 5 treatments). Appropriate IgG controls will also be employed. Tumor growth will be evaluated every 2-3 days and volumes calculated. Cohorts will be sacrificed and analyzed when the control tumor reach a volume close to 1000±200 mm³. We anticipate we will observe a 25% reduction in tumor growth in bacteria^{low} treated mice as compared to controls evaluated in 15 mice/group. In addition, at sacrifice, the levels of BAC-TA cross-reactive T-cells can be assessed via IL-10 ELISPOT. Levels of Type I and Type II TIL can be evaluated by flow cytometric analysis.

### Example 5: Bacteria Specific IL-10 Secreting T-Cells Derived from the Gut are Cross-Reactive with Tumor Antigens and Accelerate Tumor Growth in Mouse Models

This Example demonstrates that tumor antigen epitopes with elevated IL-10-secreting T-cell precursor frequencies share high sequence homology with epitopes from bacteria in the gut microbiome. Moreover, human T-cell lines generated from IL-10-inducing tumor antigen epitopes were cross-reactive to bacteria with high sequence homology, expressed cytokines and genes associated with intestinal intraepithelial lymphocytes and were of a memory phenotype. Murine T-cell lines generated from the same IL-10-inducing tumor antigen epitopes observed in humans recapitulated the phenotype of the human T-cell lines, confirming that the results observed in human T-cells were similar for murine T-cells. Significantly more TCRb were common between intestinal intraepithelial lymphocytes and tumor infiltrating lymphocytes than splenocytes and tumor infiltrating lymphocytes. This Example further shows that Pseudomonas aeruginosa-specific T-cells traffic to breast cancer and promote tumor growth. Bacterial-tumor antigen cross reactive-specific TCRb can be identified in tumor biopsies of newly diagnosed breast cancer patients. These results provide support for new methods of treating cancer and inhibiting tumor growth.

### Materials and Methods

Human subjects. Peripheral blood mononuclear cells (PBMC) from 22 female breast cancer patients and 21 female volunteer controls were collected after informed consent and cryopreserved as previously described [1]. The University of Washington Human Subjects Division approved sample collection.

Analysis of peptide and protein-specific T-cell responses. 15-20mer peptides derived from overexpressed nonmutated cancer associated proteins (Table 3), predicted to promiscuously bind human MHCII, were selected using multiple web based algorithms as previously described [2]. The peptides were constructed and purified by high-performance liquid chromatography (>90% pure; CPC Scientific). Human PBMC or mouse splenocytes were evaluated by ELISPOT for antigen-specific IFN-gamma (γ) using 10 ug/ml peptides or IL-10 using 10 g/ml peptides, 1 ug/ml human recombinant protein (Abnova), low endotoxin bovine serum albumin (BSA) (Gemini Bio-products), and 0.1 mg/ml low endotoxin bacterial lysate production as previously described [3]. All bacterial isolates were purchased from ATCC and cultured using methods established at ATCC. Cultures in log phase were lysed in sterile PBS with five rounds of freezing and thawing. Cultures were centrifuged at 14,000×g for 15 min, and the protein concentration measured in the supernatant using the BCA Protein Assay (Pierce). Individual use aliquots of lysate were stored at −80 C. ELISPOT data are presented as precursor frequency or corrected spots per well (experimental wells-unstimulated wells)

TABLE 3
Antigen and amino acid location
of the tested epitopes

| protein | start | end |
|---|---|---|
| birc5 | 13 | 30 |
| birc5 | 6 | 23 |
| birc5 | 61 | 75 |
| birc5 | 86 | 102 |
| cd105 | 99 | 115 |
| cd105 | 116 | 130 |
| cd105 | 214 | 236 |
| cd105 | 258 | 277 |
| cd105 | 302 | 319 |
| cd105 | 316 | 335 |
| cd105 | 484 | 501 |
| cd105 | 569 | 585 |
| cd105 | 603 | 625 |
| cd105 | 626 | 642 |
| cd105 | 95 | 114 |
| cdc25b | 132 | 156 |
| cdc25b | 298 | 319 |
| cdc25b | 375 | 394 |
| cdc25b | 400 | 418 |
| cdc25b | 421 | 445 |
| cdc25b | 468 | 485 |
| cdc25b | 47 | 67 |
| cdc25b | 505 | 519 |
| cdh3 | 137 | 156 |
| cdh3 | 179 | 197 |
| cdh3 | 246 | 264 |
| cdh3 | 405 | 425 |
| cdh3 | 476 | 493 |
| cdh3 | 508 | 525 |
| cdh3 | 601 | 619 |
| cdh3 | 656 | 674 |
| cdh3 | 667 | 684 |
| cdh3 | 778 | 794 |
| cdh3 | 93 | 110 |
| foxq1 | 124 | 144 |
| foxq1 | 156 | 180 |
| foxq1 | 183 | 202 |
| foxq1 | 205 | 219 |
| foxq1 | 271 | 285 |
| foxq1 | 370 | 384 |
| hif1a | 113 | 138 |
| hif1a | 168 | 183 |
| hif1a | 189 | 208 |
| hif1a | 276 | 295 |
| hif1a | 312 | 331 |
| hif1a | 340 | 359 |
| hif1a | 38 | 53 |
| hif1a | 527 | 544 |
| hif1a | 60 | 82 |
| hif1a | 771 | 785 |
| hif1a | 93 | 117 |
| mdm2 | 104 | 121 |
| mdm2 | 195 | 211 |
| mdm2 | 30 | 49 |
| mdm2 | 41 | 60 |
| mdm2 | 460 | 476 |
| mdm2 | 57 | 74 |
| mdm2 | 80 | 96 |
| mdm2 | 97 | 111 |
| pri3 | 104 | 122 |
| pri3 | 12 | 30 |
| pri3 | 124 | 142 |
| pri3 | 33 | 53 |
| satb1 | 160 | 179 |
| satb1 | 224 | 238 |
| satb1 | 240 | 259 |
| satb1 | 336 | 355 |
| satb1 | 395 | 409 |
| satb1 | 423 | 442 |
| satb1 | 514 | 528 |
| satb1 | 548 | 562 |
| satb1 | 690 | 709 |
| satb1 | 98 | 117 |
| sox2 | 193 | 210 |
| sox2 | 217 | 235 |
| sox2 | 267 | 281 |
| sox2 | 45 | 60 |
| sox2 | 91 | 107 |
| yb1 | 138 | 153 |
| yb1 | 235 | 249 |
| yb1 | 281 | 294 |
| yb1 | 50 | 68 |
| yb1 | 62 | 81 |
| yb1 | 82 | 98 |

Antigen-specific T-cell lines were generated using published methods [4], with the modification that only IL-2 was added to maintain cultures and only one in vitro stimulation was performed. The antigen-specific T-cells were a mean of 75% (range 66-90%) CD3+ T-cells, which consisted predominantly of CD4+ T-cells (mean 65%, range 58-78% of CD3+), with few CD8+ T-cells (mean 10%; range, 8-12% of CD3+). Splenocytes were cultured with a previously optimized dose of P. aeruginosa lysate (0.1 μg/ml) that had been treated to reduce endotoxin. On day 5 of culture, 20 ng/ml recombinant murine IL-2 was added. On day 8 of culture, autologous, irradiated splenocytes loaded with P. aeruginosa lysate was added 1:1 to the original T-cell culture. The P. aeruginosa specific T-cells were a mean of 75% (range 66-90%) CD3⁺ T-cells, which consisted predominantly of CD4⁺ T-cells (mean 65%, range 58-78% of CD3⁺), with few CD8⁺ T-cells (mean 10%; range, 8-12% of CD3⁺).

Epitope feature collection. Epitope position was defined as the position of the first amino acid in the epitope, divided by the length of the protein (giving a number between 0 and 1). Mutation location data was obtained from the Exome Variant Server, NHLBI GO Exome Sequencing Project (ESP) Seattle, WA, data release (ESP6500SI-V2). Homologous proteins for an epitope were obtained by querying the refseq protein database via Blast using the entire protein and filtering for bacterial, fungal and viral organisms in the human microbiome as defined in the Human Microbiome Project [5]. The Blast search settings were set with a maximum E-value of 200000 and a maximum of 10000 hits; all other search settings were default. Epitopes were not evaluated further if there was <50% identity with any microorgnisms [6].

### Murine Study Design

Animal care and use were in accordance with University of Washington IACUC guidelines. Individual mice were randomized into treatment groups by age and assigned sequentially until the study was fully enrolled. The investigators and animal caretakers were not blinded to the treatment groups, but the treatments were administered in random cage order. In vivo studies were terminated when the volume of the treated group was statistically significantly more than the control for at least two measurements. All mice in in vivo experiments were included in the data analysis. All methods are reported in accordance with ARRIVE guidelines for animal studies.

Animal model, adoptive transfer and assessment of tumor growth. Female, FVB mice (8 weeks old, median weight: 18 g, range: 16.5-18.5, Jackson Labs) FVB-Tg (C3-1-TAg) cJeg/Jeg (C3(1)-Tag) mice (6 weeks old; median weight: 18.5 g, range: 16.5-18.5 g; provided by Dr. Jeff Green, NCI) were used in this study. Tumors derived from the C3(1)Tag mouse are consistent with a basal phenotype [7]. The mouse mammary tumor cell line, M6, is derived from a spontaneous tumor from C3(1)-Tag mice (provided by Dr. Jeff Green, NCI [8]). The M6 cell line was authenticated before use and verified to express the SV40 antigen by Western blot and negligible levels of estrogen receptor by RT-PCR. For tumor challenge, 0.5×10⁶ M6 cells were implanted into the flank [3]. Some mice were allowed to develop spontaneous tumors to a volume of 500±50 mm³. Tumors were measured as previously described [2]. All tumor growth is presented as mean tumor volume (mm³±SEM).

For adoptive transfer, on day 14 of T-cell culture, 1×10⁷ P. aeruginosa-specific T-cells or naïve splenocytes were labeled with 320 μg/ml XenoLight DIR (Perkin Elmer) in 5 ml PBS then washed twice with PBS. Uptake of the dye was confirmed via flow cytometry. The cells were injected into the tail vein of each of 4-5 mice bearing 25-75 mm³ tumors. After 72 h, mice were fluorescently imaged on a Perkin Elmer IVIS imager and fluorescent output (Total flux, photons/second) was quantified on the Living Image software.

Cell phenotyping. RNA was isolated from human and murine T cells using the RNAqueous-4PCR (Life Technologies) kit according to manufacturer's instructions. RNA quantity was determined with a NanoDrop Spectrophotometer. cDNA was synthesized from 100 μg of RNA using the SuperScript III RT (Life Technologies) kit according to the manufacturer's instructions. Gene expression was assessed via TaqMan (ABI 7900HT) Real time PCR using 50 ng of cDNA and 1 pg of the specific TaqMan Gene Expression Array (Life Technologies).

Cytokine secretion was measured with the Milliplex Multiplex Assays using Luminex (Millipore Sigma) according to the manufacturer's instructions in triplicates from 72 h cell culture supernatants.

Intestinal intraepithelial and tumor infiltrating lymphocyte isolation. Small intestines were removed and carefully cleaned from their mesentery then opened longitudinally and washed of fecal contents. Intestines were then cut into 0.5-cm pieces, transferred into 50 ml conical tubes, and shaken at 250 rpm for 30 min at 37 °C in Hanks'-5 (Ca/Mg-free HBSS plus 5% FBS) with 1 mM EDTA and 1 mM DTT (Sigma-Aldrich). The cell suspensions were passed through a strainer, then over sterile gauze. Whole tumors were cut into 10 mm pieces, incubated in the manufacturer's recommended enzyme mix from the Tumor Dissociation kit (Miltenyi Biotec) then applied to the “mouse TDK1” program on the gentleMACs dissociator. Red cells were lysed. Intraepithelial and tumor infiltrating lymphocytes were selected via the CD45 magnetic bead enrichment kit (Miltenyi Biotec) according to the manufacturer's instructions or fluorescently-labeled T-cells were selected via flow cell sorting (selectively sorting on cells emitting fluorescence at the 750 nm) and evaluated via ELISPOT for bacteria-specific IL-10 secretion.

Immunophenotyping. Prior to surface staining, cells were stained with Fixable viability dye 450 (eBiosciences; 1:1000) in PBS and then washed in flow cytometry buffer. Antibodies were obtained from BD Bioscience or Biolegend. Receptor expression was documented by incubating with anti-mouse-CD3e (FITC; clone 145-2011), -CD4 (BV605; clone RM4-4), -CD8a (AlexaFluor700; clone 53-6.7), -CD44 (PE; clone IM7), -CD62L (APC; clone MEL-14) or anti-human-CD3e (FITC; clone SK7), -CD4 (APC-Cy7; clone RPAT4), -CD8a (PerCP-Cy5.5; clone SK-1), -CD45RA (BV650; clone HI100), -CCR7 (Alex700; clone 150503) or the appropriate isotype control for 1 hour at 2-8 °C protected from light. Analysis was performed on FACS Canto RUO and data analyzed using FlowJo software (BD Biosciences). Typically, 2×10⁵ events were recorded from the viability gate per sample. Results are reported as a percentage of total cell number or a percentage of a specific cell population

T-cell receptor sequencing. T-cell receptor beta chain CDR3 regions were sequenced from DNA isolated from murine intestinal intraepithelial lymphocytes, tumor infiltrating lymphocytes and spleen or human T-cell lines, PBMC or tumor biopsies by ImmunoSeq (Adaptive Biotechnologies), with primers annealing to V and J segments, resulting in amplification of rearranged VDJ segments from each cell and differential expression analyzed with the ImmunoSeq Analyzer software.

16s Amplicon Sequencing and Analysis. Frozen tumor and spleen were provided to Microbiome Insights, Inc. DNA was extracted from samples using the Qiagen MagAttract PowerSoil DNA KF Kit optimized for the Thermofisher KingFisher robot. DNA quantification and quality checks are done via Qubit. The library was preparation using the high-fidelity Phusion polymerase for amplification of 16s marker genes. The PCR reactions were normalized using the high-throughput SequalPrep 96-well Plate Kit. Samples are then pooled to make one library that was quantified accurately with the KAPA qPCR Library Quant kit. Library pools were sequenced on the Illumina MiSeq using the corresponding primer sets for prokaryotic regions.

Statistical analysis. R version 3.3.0 was used for all statistical analyses. ELISPOT count data was pre-processed according to published methods [9]. Replicates with variability in the 95% quantile were discarded, where variability is defined as the sample variance of the replicates/(median of the replicates+1). In addition, plates with 75% or more of the wells with exactly zero spots were discarded. T-tests allowing for unequal variances and linear regression (calculated using the Huber-White sandwich estimate for standard errors) were used to compare the sequence homologies, the relative epitope position, the number of non-synonymous amino acid mutations. Some comparisons used a two-tailed Wilcoxon test, generalized estimating equation (with inference drawn from a cluster-resampled bootstrap) and sign tests.

### Results

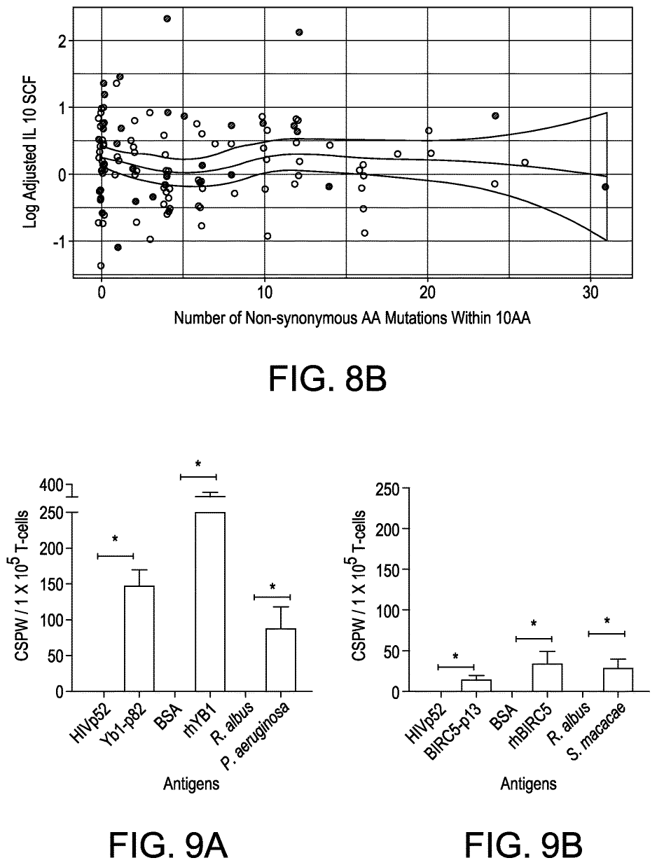

Tumor antigen epitopes with elevated IL-10-secreting T-cell precursor frequencies share high sequence homology with epitopes from bacteria in the gut microbiome. We evaluated 84 epitopes from 12 nonmutated overexpressed tumor antigens associated with cancer stem cells and demonstrated that certain epitopes could be identified from each protein which preferentially elicit an IL-10 response from multiple volunteer or cancer donors. There was no difference observed in the magnitude of the IL-10 response associated with cancer status. Indeed, we found high levels of cancer stem cell antigen specific IL-10 in the peripheral blood of individuals who did not have cancer (FIG. 7A). For some epitopes, upwards of 1:1000 T-cells in the peripheral blood was specific for a particular tumor associated antigen epitope.

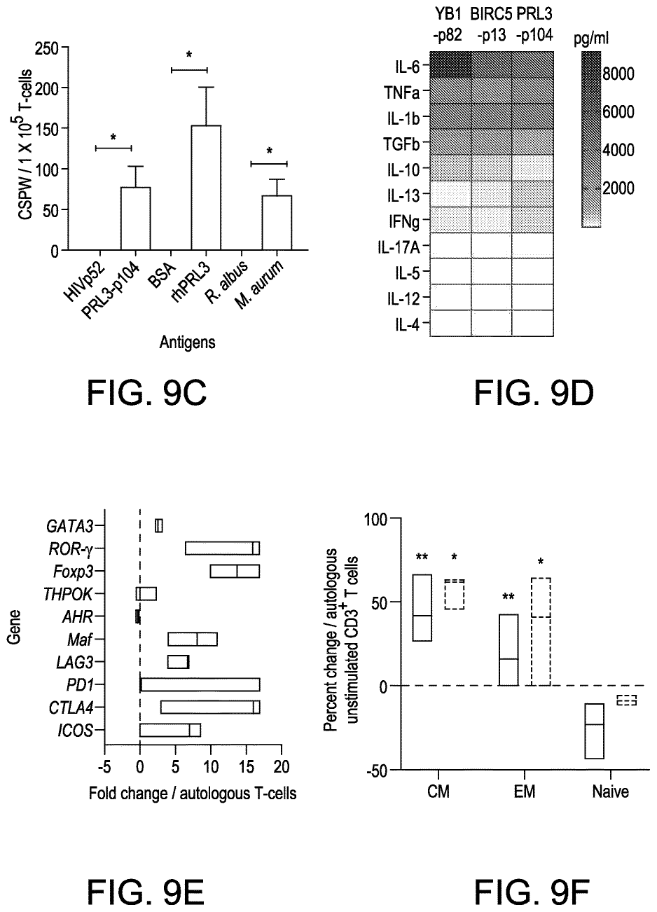

We questioned if there were any characteristics that would specifically define an IL-10-inducing tumor antigen associated epitope. Since there was no difference between volunteer or cancer donors, we combined all the data and ranked the epitopes by IL-10-secreting T-cell precursor frequencies and compared epitopes from the top (median, 1:8000 antigen specific T-cells; range, 1:1,200-1:12,000) and bottom quintiles (median, 1:98,000 antigen-specific T-cells; range, 1:75,000-1:2.5×10⁶). There was no significant association with the IL-10 response generated by that epitope and the location of the epitope in the protein (FIG. 8A) or the number of commonly reported mutations within 10 amino acids of an epitope (FIG. 8B). Location of the protein in the cell has been shown to be associated with immunogenicity, however, we did not evaluate cellular location as a potential factor associated with IL-10 response since most of the antigens in this study were intracellular. We evaluated potential sequence homologies for all epitopes with microorganisms found in the gut microbiome. The epitopes in the top quintile (median, 110 species; range, 0-5,582) were homologous with more bacteria in the gut microbiome than the epitopes in the bottom quintile (median, 48 species; range, 0-184; p=0.012; FIG. 7B). There was no difference in the number of fungal (p=0.75; FIG. 7C) or viral species (p=0.37; FIG. 7D) between the top and bottom quintiles. We found 11 bacterial species with homologies to more than ten tumor antigen epitopes with at least 86% of the donors evaluated responding to those epitopes by secreting IL-10. (Table 4).

TABLE 4
BAC-TA IL-10 stimulating CD4 T-cell epitopes that are
commonly detected in the majority of individuals tested.

| | No. of | Proportion |
|---|---|---|
| Species | Homologies to TA | Responders |
| Escherichia coli | 30 | 0.93 |
| Enterococcus faecalis | 17 | 0.93 |
| Pseudomonas fluorescens | 16 | 0.91 |
| Pseudomonas putida | 16 | 0.91 |
| Pseudomonas aeruginosa | 15 | 0.95 |
| Shigella dysenteriae | 14 | 0.91 |
| Enterobacter cloacae | 13 | 0.93 |
| Lactococcus lactis | 12 | 0.91 |
| Clostridium perfringens | 11 | 0.91 |
| Klebsiella pneumoniae | 11 | 0.91 |
| Staphylococcus aureus | 11 | 0.86 |

Human T-cell lines generated from IL-10-inducing tumor antigen epitopes were cross-reactive to bacteria with high sequence homology, expressed cytokines and genes associated with intestinal intraepithelial lymphocytes and were of a memory phenotype. Three different IL-10-selective epitopes with a high IL-10-secreting precursor frequency of antigen-specific T-cells in PBMC were used for evaluation. The median level of circulating Yb1-p82 specific IL-10 secreting T-cells was 1:56,000 (range 1:600-1:80,000) of PBMC from 40% of donors. T-cells from 78% of donors secreted IL-10 in response to PRL3-p104 at a median precursor frequency of 1:9000 (range 1:550-1:62,000). Ninety percent of donor PBMC responded to BIRC5-p13 by secreting IL-10 at a median precursor frequency of 1:9000 (range 1:1400-1:80,000).

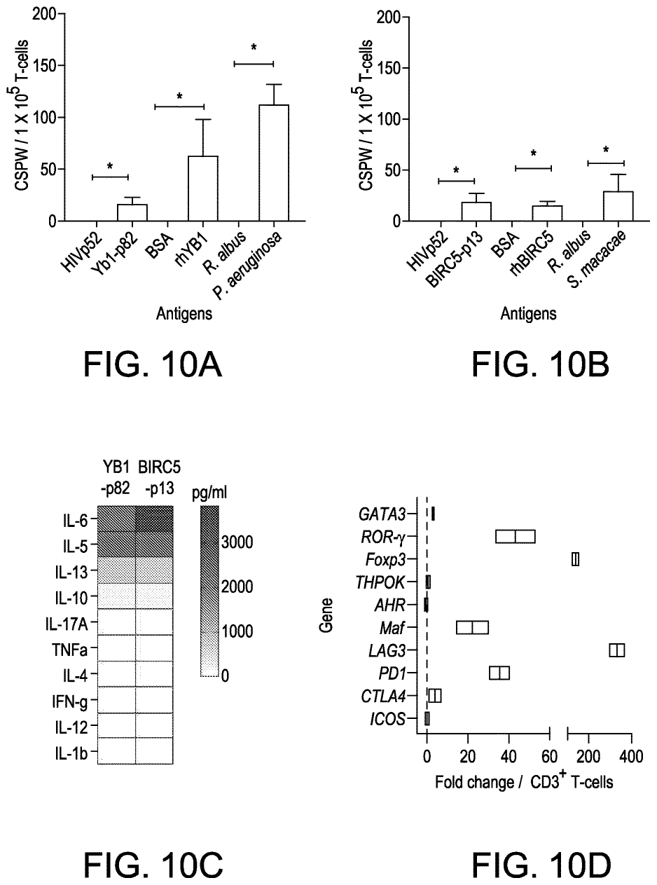

Antigen-specific T-cell lines were generated with each of these epitopes. Yb1-p82 is 58% homologous to Pseudomonas (P.) aeruginosa and shares no homology with Ruminococcus (R.) albus. Yb1-p82-specific T-cells confirmed to be epitope and protein specific exhibited significant IL-10 secretion when incubated with P. aeruginosa lysate (mean, 89.8±28 CSPW) as compared to the negative lysate control R. albus (mean, 0 CSPW; p=0.029; FIG. 9A). A similar result was observed for PRL3-p104- and BIRC5-p13-specific T-cells. PRL3-p104-specific T-cells demonstrated significant IL-10 secretion when incubated with M. aurum lysate (63% homologous; mean, 68.3±19 CSPW) as compared to the R. albus lysate (mean, 0 CSPW; p=0.029; FIG. 9B). BIRC5-p13-specific T-cells also exhibited significant IL-10 secretion when stimulated with S. macacae lysate (50% homologous; mean, 30±9.6 CSPW) as compared to R. albus lysate (mean, 0 CSPW; p=0.029; FIG. 9C).

To discern the phenotype of the bacteria-tumor antigen cross-reactive T-cells, we evaluated antigen-specific cytokine secretion and expression of markers associated with specific T-cell lineages. As expected, the bacterial-tumor antigen cross reactive epitopes generated IL-10 secretion from the T-cells in all donors (mean, 1,954 μg/ml; range 1,000-2,705 μg/ml), but very low levels of IL-4 (mean 10 μg/ml) and IL-5 (95 μg/ml). The IL-10 inducing epitopes also secreted high levels of IL-6 (mean, 41,510 μg/ml; range 38,812-45,272 μg/ml) and higher levels of TNF-α (mean, 28,442 μg/ml; range, 26,062-29,797 pg/ml), IL1-β (mean, 12,791 μg/ml, range 12, 505-12,965 μg/ml) as compared to autologous CD3+ T-cells (FIG. 9D). The genes c-Maf, PD1, ICOS, FOXP3, CCR9, ROR-g, LAG3 and CTLA4 were expressed greater than a mean of 4-fold in the bacterial-tumor antigen cross reactive T-cells as compared to autologous unstimulated CD3⁺ selected T-cells. AHR, ThPOK and GATA3 were expressed at lower levels in the T-cell lines (FIG. 9E), suggesting an intestinal intraepithelial-like lymphocyte phenotype. Significantly more central and effector memory cells than naïve cells in both T-cell compartments were observed in the bacterial-tumor antigen cross-reactive T-cells as compared to autologous unstimulated CD3⁺ selected T-cells (p<0.05 for all; FIG. 9F).

Murine T-cell lines generated from the same IL-10-inducing tumor antigen epitopes observed in humans recapitulated the phenotype of the human T-cell lines. We confirmed that the results observed in human T-cells was also similar for murine T-cells. The high precursor frequency observed for the three IL-10-selective epitopes in human PBMC was also observed for two of the three same epitopes in mouse splenocytes. The median level of circulating Yb1-p82 specific IL-10 secreting T-cells was 1:10,000 (range 1:10,000-1:71,000) of splenocytes from 30% of donors. No PRL3-p104-specific IL-10-secreting cells were observed in any donor. Sixty percent of the mice responded to BIRC5-p13 by secreting IL-10 at a median precursor frequency of 1:7000 (range 1:4000-1:76,000).

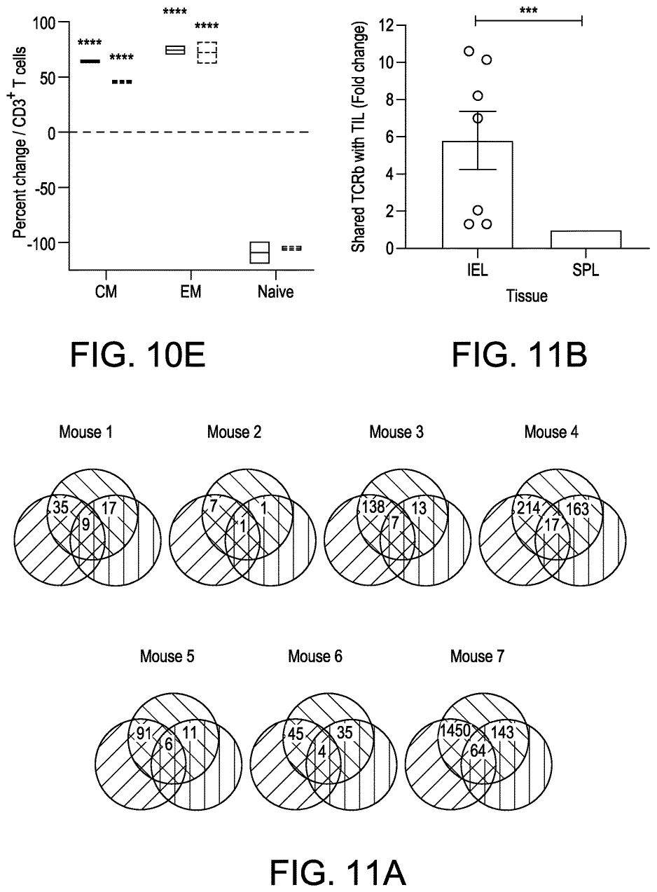

Yb1-p82-specific T-cells confirmed to be epitope and protein specific exhibited significant IL-10 secretion when incubated with P. aeruginosa lysate (mean, 113.5±18 CSPW) as compared to the negative lysate control R. albus (mean, 0 CSPW; p=0.029; FIG. 10A). Similarly, BIRC5-p13-specific T-cells also exhibited significant IL-10 secretion when stimulated with S. macacae lysate (mean, 30±15 CSPW) as compared to R. albus lysate (mean, 0 CSPW; p=0.029; FIG. 10B).

The IL-10 inducing epitopes also secreted high levels of IL-6 (mean 3022 μg/ml) and higher levels of IL-10 (mean, 175 μg/ml), IL-5 (mean 2248 μg/ml) and IL-13 (mean 785 μg/ml) as compared to autologous CD3+ T-cells (FIG. 10C). The genes c-Maf, PD1, FOXP3, ROR-g, and LAG3 were expressed greater than a mean of 20-fold in the bacterial-tumor antigen cross reactive T-cells as compared to unstimulated CD3⁺ selected T-cells. AHR, ThPOK, ICOS, GATA3 and CTLA4 were expressed at lower levels in the T-cell lines (FIG. 10D). Significantly more central and effector memory cells than naïve cells in both T-cell compartments were observed in the bacterial-tumor antigen cross-reactive T-cells as compared to autologous unstimulated CD3⁺ selected T-cells (p<0.05 for all; FIG. 10E).

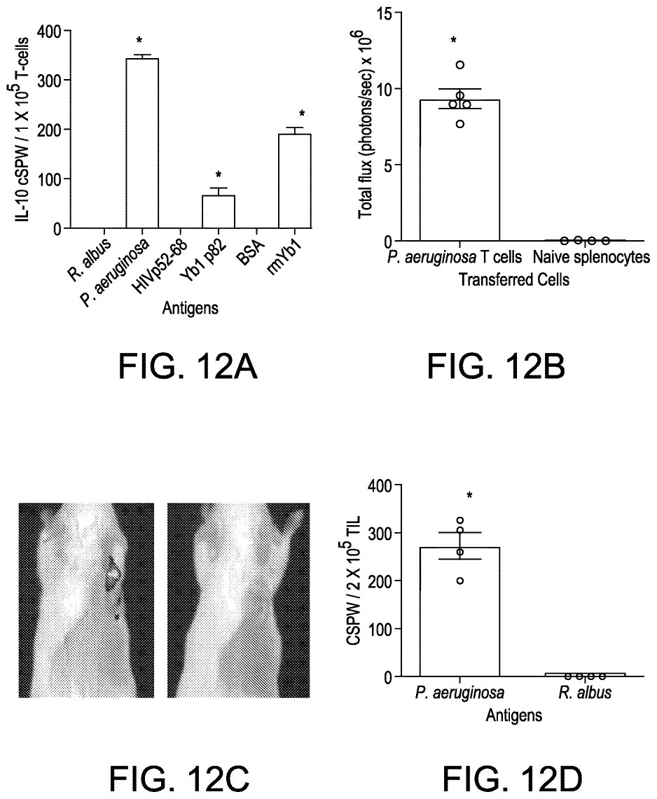

Significantly more TCRb were common between intestinal intraepithelial lymphocytes and tumor infiltrating lymphocytes than splenocytes and tumor infiltrating lymphocytes. To explore the source of the bacterial-tumor antigen cross reactive T-cells, we evaluated TCRb sequences among corresponding intestinal intraepithelial lymphocytes, splenocytes and tumor infiltrating lymphocytes from mice who developed spontaneous breast cancer. The median number of shared TCRb between intestinal intraepithelial lymphocytes and tumor infiltrating lymphocytes was 91 (range, 7-1450) and between splenocytes and tumor infiltrating lymphocytes was 17 (range, 1-143; FIG. 11A). There was a significantly greater number of shared TORb between intestinal intraepithelial lymphocytes and tumor infiltrating lymphocytes than splenocytes and tumor infiltrating lymphocytes (FIG. 11B; p=0.0006).

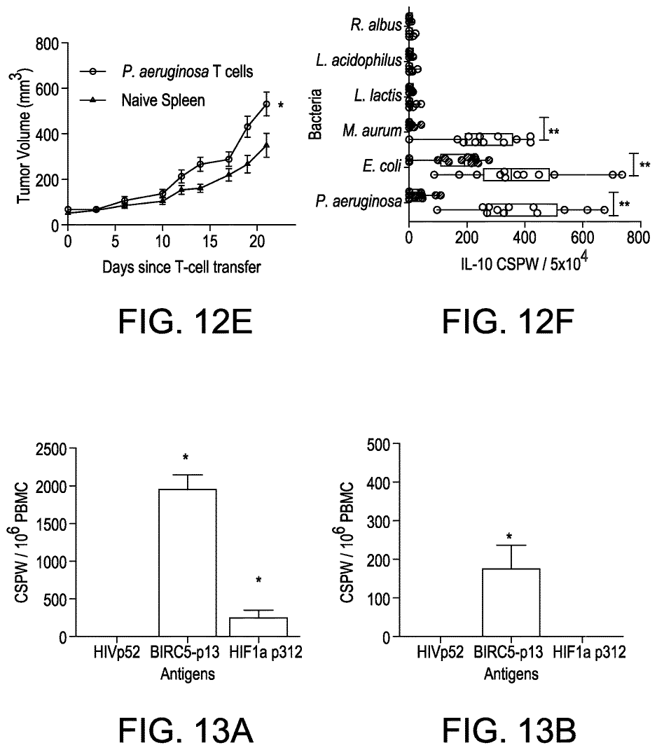

Pseudomonas aeruginosa-specific T-cells traffic to breast cancer and promote tumor growth. Since we had demonstrated that peptide-specific T-cells could cross-react with bacteria with high homology, we next questioned if bacterial-specific T-cells could cross-react with a high homology epitope. Using P. aeruginosa as a representative bacterium, we generated an antigen-specific T-cell line. P. aeruginosa-specific T-cells were cross-reactive with the high homology epitope Yb1-p82. Significant IL-10 secretion was generated by Yb1-p82, compared to an irrelevant peptide HIVp52 (p=0.029; FIG. 12A) or recombinant Yb1 protein compared to an equivalent dose of an irrelevant protein BSA (p=0.029; FIG. 12A). We confirmed that the Yb1 peptide and protein were being presented in the context of MHCII since significantly less IL-10 was detected from cells that had been treated with both an MHCII blocking antibody and Yb1-p82 (p=0.029) or rYb1 (p=0.029).

Next, we questioned if the P. aeruginosa-specific T-cells would traffic to an established tumor. Adoptive transfer with P. aeruginosa-specific T cells resulted in significantly more antigen-specific T-cells in the tumors as compared to adoptive transfer of naïve splenocytes (p=0.029; FIGS. 12B and 12C). Notably, the P. aeruginosa-specific T-cells were specifically detected in the tumor and not observed in secondary lymphoid organs such as the spleen. We confirmed that the TIL were specific for P. aeruginosa. Significantly higher magnitude IL-10 secreting cells were observed from TIL stimulated with P. aeruginosa (272±28 cSPW) as compared to R. albus (0 cSPW; p=0.029; FIG. 12D). P. aeruginosa-specific T-cells promoted tumor growth. Tumors from mice receiving P. aeruginosa-specific T-cells were significantly larger (554±62 mm³) than tumors from mice receiving naïve splenocytes (245±42 mm3; p=0.02; FIG. 12E).

Antigen-specific T cells cross-reactive between IL-10-selective self-epitopes and bacteria were present in spontaneous breast cancer. Significantly more IL-10 secreting cells were detected in the TIL stimulated with lysate from bacteria with high homologies to tumor antigens, P. aeruginosa (380±47cSPW), E. coli (435±55 cSPW) or M. aurum (261±34 cSPW), as compared to spleen (p<0.01 for all; FIG. 12F). Sequencing for bacteria in the tumors did not reveal the presence of any of these species. There was no significant IL-10 secretion in response to the low tumor antigen homology bacteria species, L. lactis, L. acidophilus or R. albus in the TIL or spleen (FIG. 12F).

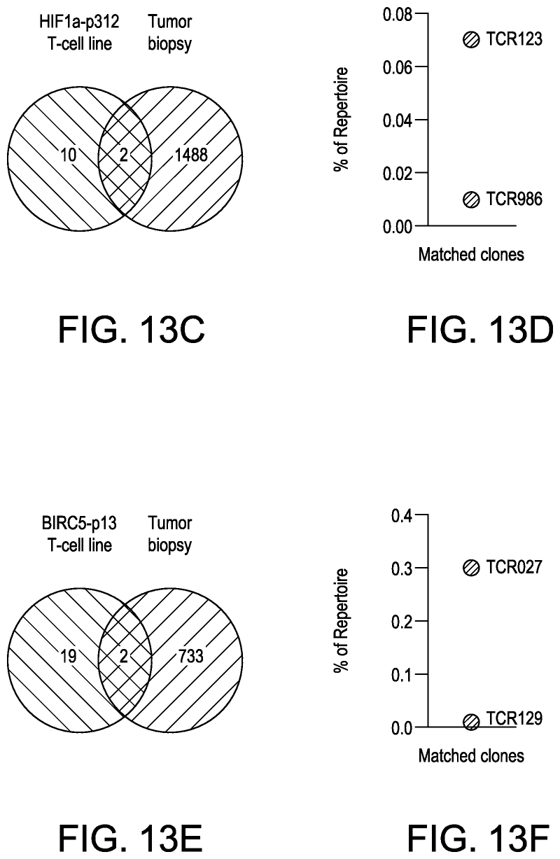

Bacterial-tumor antigen cross reactive-specific TCRb can be identified in tumor biopsies of newly diagnosed breast cancer patients. We observed a significant IL-10 response to the BIRC5-p13 epitope in PBMC of newly diagnosed breast cancer Donor 1 (mean, 178±59 cSPW, p=0.02; FIG. 13A) and Donor 2 (mean, 1968±174 cSPW, p=0.02; FIG. 13B). Additionally, we observed a significant IL-10 response to the HIFa-p312 epitope in Donor 1 (mean, 260±90 cSPW, p=0.02; FIG. 13A). When we generated epitope-specific T-cell lines, we observed 10 distinct TCRb sequences for HIF1a-p312 in Donor 1 (FIG. 13C). Of the 1,488 TCRb sequences observed in the corresponding tumor biopsy, two sequences were also found in the HIF1a-p312-specific T-cell line, CASSEEAGGYNEQFF (SEQ ID NO: 1; TCR096; representing 0.01% of the tumor TCRb repertoire) and CASSLEGSSNF (SEQ ID NO: 2; TCR123; representing 0.07% of the tumor TORb repertoire; FIG. 13D). None of the 11 TCRb sequences observed in the BIRC5-p13-specific T-cell line generated from Donor 1's PBMC were found in the tumor biopsy of Donor 1. Nineteen distinct TCRb sequences were observed for the BIRC5-p13 T-cell line in Donor 2 (FIG. 13E). Of the 733 TCRb sequences in the corresponding tumor biopsy, two sequences were also found in the BIRC5-p13-specific T-cell line, CSASKGLAISTDTQYF (SEQ ID NO: 3; TCR027; representing 0.3% of the tumor TCRb repertoire) and CASSIWVGGAHGYTF (SEQ ID NO: 4; TCR129; representing 0.01% of the tumor TCRb repertoire; FIG. 13E).

### REFERENCES

1. Disis, M. L., et al., J Immunol Methods, 2006. 308(1-2): p. 13-8.
2. Park, K. H., et al., Cancer Res, 2008. 68(20): p. 8400-9.
3. Cecil, D. L., et al., Cancer Res, 2014. 74(10): p. 2710-8.
4. Dang, Y., et al., Clin Cancer Res, 2007. 13(6): p. 1883-91.
5. Human Microbiome Project, C., Nature, 2012. 486(7402): p. 207-14.
6. Aalberse, R. C., J Allergy Clin Immunol, 2000. 106(2): p. 228-38.
7. Herschkowitz, J. I., et al., Genome Biol, 2007. 8(5): p. R76.
8. Holzer, R. G., et al., Breast Cancer Res Treat, 2003. 77(1): p. 65-76.
9. Moodie, Z., et al., Cancer Immunol Immunother, 2010. 59(10): p. 1489-501.

Throughout this application various publications are referenced. The disclosures of these publications in their entireties are hereby incorporated by reference into this application in order to describe more fully the state of the art to which this invention pertains.

From the foregoing it will be appreciated that, although specific embodiments of the invention have been described herein for purposes of illustration, various modifications may be made without deviating from the spirit and scope of the invention. Accordingly, the invention is not limited except as by the appended claims.

1. A method of enhancing immunity to a tumor antigen and/or slowing tumor growth in a subject, the method comprising: administering to the subject an effective amount of the composition of claim 17.

2. (canceled)

3. (canceled)

4. (canceled)

5. (canceled)

6. The method of claim 1, wherein the composition is free of bacteria exhibiting greater than 50% homology to tumor antigens identified in a blood sample obtained from the subject.

7. The method of claim 1, wherein the composition is administered weekly for six weeks.

8. The method of claim 1, wherein the effective amount is sufficient to effect a two-fold increase in gut microbiome bacteria^{low}, and/or a significant decrease in bacterial-tumor antigen (BAC-TA) cross-reactive memory T-cells, in a biological sample obtained from the subject.

9. The method of claim 1, wherein the method enhances efficacy of a cancer treatment, wherein the cancer treatment comprises immunotherapy and/or chemotherapy, and wherein the composition is administered prior to and/or concomitantly with the immunotherapy or chemotherapy.

10. (canceled)

11. The method of claim 10, wherein the immunotherapy comprises administration of immune checkpoint inhibitor therapy, vaccine therapy, cytokine therapy, oncolytic virus therapy, and/or monoclonal antibodies.

12. The method of claim 11, wherein the monoclonal antibodies comprise anti-HER2, anti-EGFR, and/or anti-VEGF antibodies.

13. The method of claim 10, wherein the chemotherapy comprises administration of paclitaxel, cisplatin, carboplatin, pemtrexed, Adriamycin, or Cytoxan.

14. (canceled)

15. (canceled)

16. The method of claim 1, wherein the composition comprises 1-5×10⁹ CFU of bacteria^{low}.

17. A composition comprising a plurality of bacteria that exhibit less than 50% homology to non-mutated tumor antigens (bacteria^{low}), wherein the plurality of bacteria^{low} are selected from: Lactobacillus acidophilus, Ruminococcus albus, Ruminococcus gnavus, Dorea formicigenerans, Eubacterium siraeum, Bacteroides stercoris, Parabacteroides distasonis, Bifidobacterium breve, Lactobacillus delbrueckii, Odoribacter splanchnicus, Akkermansia muciniphila, Desulfovibrio piger, Mitsuokella multacida, Bifidobacterium catenulatum, Staphylococcus saprophyticus, Bacteroides vulgatus, Bifidobacterium pseudocatenulatum, Bifidobacterium angulatum, Bifidobacterium bifidum, Campylobacter hominis, Clostridium leptum, Clostridium methylpentosum, Coprococcus catus, Eubacterium ventriosum, Ruminococcus bromii, Victivallis vadensis, Bacteroides eggerthii, Bacteroides helcogenes, Granulicatella adiacens, Peptoniphilus harei, and
wherein the plurality of bacteria^{low} comprises at least four bacteria selected from: R. albus, L. acidophilus, A. muciniphila, B. breve, O. splanchnicus and P. distasonis.

18. (canceled)

19. (canceled)

20. (canceled)

21. The composition of claim 17, which is substantially free of bacteria that exhibit greater than 50% homology to tumor antigens.

22. A method of ameliorating an autoimmune inflammatory response in a subject, the method comprising: administering to the subject an effective amount of a composition of claim 30.

23. (canceled)

24. The method of claim 22, wherein the plurality of bacteria^{high} exhibit at least 50% homology to self-proteins identified in a blood and/or stool sample obtained from the subject.

25. The method of claim 22, wherein the composition is administered weekly for six weeks to one year.

26. The method of claim 22, wherein the effective amount is sufficient to effect a two-fold increase in gut microbiome bacteria^{high} in a biological sample obtained from the subject.

27. The method of claim 22, wherein the method elicits a type 2 immune response.

28. The method of claim 22, wherein the composition comprises 1-5×10⁹ CFU of bacteria^{high}.

29. The method of claim 1, wherein the administration is oral, rectal, or intragastric.

30. A composition comprising a plurality of bacteria that exhibit at least 50% amino acid sequence homology to self-proteins (bacteria^{high}), wherein the plurality comprises at least four species of bacteria^{high} selected from: Escherichia coli, Enterococcus faecalis, Pseudomonas fluorescens, Pseudomonas putida, Pseudomonas aeruginosa, Shigella dysenteriae, Enterobacter cloacae, Lactococcus lactis, Clostridium perfringens, Klebsiella pneumoniae, Staphylococcus aureus, Enterococcus faecium, Rhodopseudomonas palustris, Shigella boydii, Shigella sonnei, Finegoldia magna, Clostridium asparagiforme, Collinsella aerofaciens, Fusobacterium ulcerans, Aerococcus viridans, Bacteroides ovatus, Clostridium botulinum, Enterococcus gallinarum, Eubacterium limosum, Faecalibacterium prausnitzii, Holdemania filiformis, Lactobacillus plantarum, Lactobacillus reuteri, Marvinbryantia formatexigens, Megamonas hypermegale, Streptococcus agalactiae, Streptococcus anginosus, Streptococcus dysgalactiae, and Streptococcus infantarius.

31. (canceled)

32. (canceled)

33. The composition of claim 30, wherein the plurality comprises 6-10 species of bacteria^{high}.


SEQUENCE LISTING (4 sequences)

SEQ ID NO: 1 - protein, 15 aa, synthetic construct
CASSEEAGGYNEQFF

SEQ ID NO: 2 - protein, 11 aa, synthetic construct
CASSLEGSSNF

SEQ ID NO: 3 - protein, 16 aa, synthetic construct
CSASKGLAISTDTQYF

SEQ ID NO: 4 - protein, 15 aa, synthetic construct
CASSIWVGGAHGYTF